SEQUENCE LISTING (2 sequences)

SEQ ID NO: 1 - DNA, 5912 bp, Homo sapiens
atgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttg

SEQ ID NO: 2 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

(19) United States
(12) Patent Application Publication
(10) Pub. No.: US 2021/0278418 A1
(43) Pub. Date: Sep. 9, 2021

(54) METHOD FOR TREATING ASTHMA OR ALLERGIC DISEASE

(71) Applicant: THE CHIDLREN'S MEDICAL CENTER CORPORATION, Boston, MA (US)

(72) Inventors: Talal Amine CHATILA, Belmont, MA (US); Hani HARB, Brookline, MA (US); Mingcan XIA, Boston, MA (US); Amir MASSOUD, Tehran (IR)

(73) Assignee: THE CHIDLREN'S MEDICAL CENTER CORPORATION, Boston, MA (US)

(21) Appl. No.: 16/979,382

(22) PCT Filed: Mar. 15, 2019
(86) PCT No.: PCT/US19/22493

§ 371 Date: Sep. 9, 2020

Related U.S. Application Data
(60) Provisional application No. 62/643,476, filed on Mar. 15, 2018.
(60) Provisional application No. 62/652,630, filed on Apr. 4, 2018.
(60) Provisional application No. 62/659,379, filed on Apr. 18, 2018.

(51) Int. Cl. G01N 33/68; A61K 31/7105; A61K 31/713

(52) U.S. Cl. CPC G01N 33/6893; A61K 31/7105; G01N 2800/52; G01N 2800/122; G01N 2800/24; A61K 31/713

(57) ABSTRACT

Described herein are methods and compositions for treating asthma or an allergic disease. Aspects of the invention relate to administering to a subject an agent that targets Notch4. In one embodiment, the agent is an anti-Notch4 antibody.

### CROSS REFERENCE TO RELATED APPLICATIONS

This application is a 35 U.S.C. § 371 National Phase Entry Application of International Application No. PCT/US2019/022493 filed Mar. 15, 2019, which designates the U.S. and claims the benefit under 35 U.S.C. § 119(e) of U.S. Provisional Application Nos. 62/643,476 filed on Mar. 15, 2018, U.S. 62/652,630 filed Apr. 4, 2018 and U.S. 62/659,379 filed Apr. 18, 2018, the contents of each of which are incorporated herein by reference in their entireties.

### GOVERNMENT SUPPORT

This invention was made with Government support under Grant Nos 2R01AI065617 and R01AI115699 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Mar. 28, 2019, is named 701039-091780WOPT_SL.txt and is 25,709 bytes in size.

### BACKGROUND

Exposure to traffic-related particulate matter (PM) promotes asthma and allergic diseases. However, the precise cellular and molecular mechanisms by which PM exposure acts to mediate these effects remain unclear. An understanding of cellular targets and signaling pathways critical for the augmentation of allergic airway inflammation induced by ambient ultra fine particles (UFP) is essential for developing therapeutics to treat or prevent asthma and allergic diseases.

### SUMMARY

The invention described herein is related, in part, to the discovery that ultra fine particles exacerbate allergic airway inflammation by promoting a Jag1-Notch4-dependent interaction between Alveolar Macrophages and Allergen-Specific T cells, leading to augmented Th cell differentiation. Accordingly, one aspect of the invention described herein provides a method for treating asthma or an allergic disease, comprising administering to a subject having asthma or an allergic disease an effective amount of an agent that inhibits Notch4.

Another aspect of the invention described herein provides a method for treating asthma or an allergic disease, comprising (a) identifying a subject having asthma or an allergic disease; and (b) administering an effective amount of an agent that inhibits Notch4 to the subject.

Another aspect of the invention described herein provides a composition for the treatment of asthma or an allergic disease, the composition comprising an agent that inhibits Notch4 and a pharmaceutically acceptable carrier. In one embodiment of any aspect, the composition is formulated for inhaled administration.

In one embodiment of any aspect, the asthma is selected from the list consisting of allergic asthma, asthma without allergies, aspirin exacerbated respiratory disease, exercise induced asthma, cough variant, and occupational asthma.

In one embodiment of any aspect, the allergic disease is selected from the list consisting of allergic rhinitis, sinusitis, otitis media, atopic dermatitis, urticaria, angioedema, and anaphylaxis.

In one embodiment of any aspect, the agent that inhibits Notch4 is selected from the group consisting of a small molecule, an antibody, a peptide, a genome editing system, an antisense oligonucleotide, and an RNAi.

In one embodiment of any aspect, the antibody is a humanized antibody.

In one embodiment of any aspect, the RNAi is a microRNA, an siRNA, or a shRNA.

In one embodiment of any aspect, inhibiting Notch4 is inhibiting the expression level and/or activity of Notch4. In one embodiment of any aspect, the expression level and/or activity of Notch4 is inhibited by at least 50%, at least 60%, at least 70%, at least 80%, at least 90%, or more as compared to an appropriate control.

In one embodiment of any aspect, Notch4 is inhibited on T regulatory cells.

In one embodiment of any aspect, the method further comprises administering at least one additional anti-asthma therapeutic. In one embodiment of any aspect, the method further comprises administering at least one additional anti-allergic disease therapeutic.

One aspect of the invention described herein provides a method for preventing asthma or an allergic disease, comprising administering to a subject at risk of having asthma or an allergic disease an agent that inhibits Notch4. In one embodiment of any aspect, the method further comprises, prior to administering, identifying a subject at risk of having asthma or an allergic disease.

Another aspect of the invention describe herein provides a method for identifying a subject at risk of having asthma or an allergic disease comprising, (a) obtaining a biological sample from the subject; (b) measuring the level of Notch4 in the biological sample, wherein the subject is at risk of having asthma or an allergic disease if the level of Notch is increased as compared to a reference level; and (c) administering an agent that inhibits Notch4 to a subject at risk.

In one embodiment of any aspect, the level of Notch4 is increased by at least 2-fold, at least 3-fold, at least 4-fold, at least 5-fold, at least 6-fold, at least 7-fold, at least 8-fold, at least 9-fold, at least 10-fold, or more as compared to a reference level.

One aspect of the invention describe herein provides a method of determining the efficacy of a therapeutic in the treatment of a subject diagnosed with asthma or an allergic disease comprising, (a) determining a first level of Notch4 expression or activity in a sample provided by the subject diagnosed with asthma or an allergic disease prior to the administration of a therapeutic; (b) determining a second level of Notch4 expression or activity in a sample provided by the patient after administration of the therapeutic; and (c) comparing said first and second levels of Notch4 expression or activity, wherein the therapeutic is considered effective if said second level of Notch4 expression or activity is lower than said first level, and wherein the therapeutic administered in (b) is ineffective if said second level of Notch4 expression is the same as or higher than said first level. In one embodiment, the therapeutic is an agent that inhibits Notch4.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with asthma or an allergic disease. The term “treating” includes reducing or alleviating at least one adverse effect or symptom of an asthma or an allergic disease (e.g., inflamed airway). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state or disorder (e.g., asthma or an allergic disease) does not occur due to the actions of the methodology (such as, for example, administration of an agent that inhibits Notch4, or a composition described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not treated with a composition comprising a microbial consortium as described herein).

As used herein, the term “administering,” refers to the placement of a therapeutic (e.g., an agent that inhibits Notch4) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., asthma or an allergic disease. A subject can be male or female. A subject can be a child (e.g., less than 18 years of age), or an adult (e.g., greater than 18 years of age).

A subject can be one who has been previously diagnosed with or identified as suffering from or having a disease or disorder in need of treatment (e.g., asthma or an allergic disease) or one or more complications related to such a disease or disorder, and optionally, have already undergone treatment for the disease or disorder or the one or more complications related to the disease or disorder. Alternatively, a subject can also be one who has not been previously diagnosed as having such disease or disorder (e.g., asthma or an allergic disease) or related complications. For example, a subject can be one who exhibits one or more risk factors for the disease or disorder or one or more complications related to the disease or disorder or a subject who does not exhibit risk factors.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit Notch4, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

Methods and compositions described herein require that the levels and/or activity of Notch4 are inhibited. As used herein, Neurogenic locus notch homolog 4, also known as “Notch4” refers to a type I transmembrane protein, which is a member of a family that share structural characteristics, including an extracellular domain consisting of multiple epidermal growth factor-like (EGF) repeats, and an intracellular domain consisting of multiple different domain. Notch4 sequences are known for a number of species, e.g., human Notch4 (NCBI Gene ID: 4855) polypeptide (e.g., NCBI Ref Seq NP 004548.3) and mRNA (e.g., NCBI Ref Seq NM_004557.3). Notch4 can refer to human Notch4, including naturally occurring variants, molecules, and alleles thereof. Notch4 refers to the mammalian Notch4 of, e.g., mouse, rat, rabbit, dog, cat, cow, horse, pig, and the like. The nucleic sequence of SEQ ID NO: 1 comprises a nucleic sequence which encodes Notch4.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an asthma or an allergic disease, or a biological sample that has not been contacted with an agent disclosed herein).

As used herein, an “appropriate control” refers to an untreated, otherwise identical cell or population (e.g., a patient who was not administered an agent described herein, or was administered by only a subset of agents described herein, as compared to a non-control cell).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

This application file contains at least one drawing executed in color. Copies of this patent application publication with color drawings will be provided by the Office upon request and payment of the necessary fee.

FIGS. 1A-1G present data that show AM differentially uptake nanoparticles and highly express Jag1 in response to UFP. FIG. 1A and FIG. 1B, Flow cytometric analysis of the uptake of fluorescent nanoparticles by different lung cell populations in mice subjected to OVA+UFP-induced allergic airway inflammation. FIG. 1C, Bar graph presentation of the distribution of nanoparticles among lung macrophages (AM and IM), dendritic cells (DC) and neutrophils (Neu). FIG. 1D and FIG. 1E, Relative fold changes in Jag1 transcripts, quantitated by RT-PCR (FIG. 1D), and flow cytometric analysis of Jag1 expression (FIG. 1 E), in lung APCs purified from either Il4ra^{R576} or Il4ra^{R576}Lyz2^{Cre}Ahr^{Δ/Δ} mice and treated with PBS or UFP (10 μg/ml). 1F and 1G, Relative fold changes in Jag1 transcripts (FIG. 1D), and Jag1 expression (FIG. 1E), in lung APCs purified from either 14ra^{R576} or Il4ra^{R576}Lyz2^{Cre}Jag1^{Δ/Δ} mice and treated with PBS or UFP, as described for FIGS. 1, D and E. Results are representative of 2 independent experiments. N=3 mice/group. *p<0.05; **p<0.01; ***p<0.001, ***p<0.0001 by one-way ANOVA with post-test analysis (panels FIG. 1C, FIG. 1D and FIG. 1F) or Student's unpaired two-tailed t test (panel FIG. 1F, CD11c⁺ DC group comparison).

FIGS. 2A-2B present data that show AM support UFP-dependent upregulation of Th cell cytokine production by allergen-specific CD4⁺ T cells in a Jag1-dependent manner. FIG. 2A, Representative flow cytometric analysis of IL-4, IL-13, IL-17 and IFN-γ cytokine production by naive Il4ra^{R576}CD4⁺DO11.10⁺Rag2^{-/-} T cells co-cultured with FACS-purified AM isolated from l4ra^{R576} or Il4ra^{R576}Lyz2^{Cre}Jag1^{Δ/Δ} mice with OVA₃₂₃₋₃₃₉ peptide in the presence of UFP (10 μg/mL). Cytokine expression was analyzed in gated CD4⁺Foxp3⁻ T cell. FIG. 2B, Frequencies of CD4⁺Foxp3⁻ T cells expressing the respective cytokine upon co-culture with AM that have been either sham treated (PBS) or pulsed with OVA323-339 peptide, alone or in the presence of UFP. Results are representative of 3 independent experiments. *P<0.05, **P<0.01, ***P<0.001, and ****P<0.0001, two-way ANOVA with post-test analysis.

FIGS. 3A-3J present data that show UFP skews AM-dependent iTreg cell differentiation towards Th2/17 cell like phenotypes in Jag1-dependent manner. FIG. 3A, Representative flow cytometric analysis of the frequencies of CD4⁺Foxp3⁺ iTreg cells and their expression of IL-4, IL-13, IL-17 and IFN-γ upon co-culture of naive Il4ra^{R576}CD4⁺DO11.10⁺Rag2^{-/-} T cells with FACS-purified AM isolated from l4ra^{R576} or Il4ra^{R576}Lyz2^{Cre}Jag1^{Δ/Δ} mice pulsed with OVA₃₂₃₋₃₃₉ peptide in the presence of UFP. FIG. 3B, Frequencies of Foxp3⁺ iTreg cells and subgroups thereof expressing the respective cytokine that have been either sham treated (PBS) or pulsed with OVA₃₂₃₋₃₃₉ peptide, alone or in the presence of UFP. Results are representative of 3 independent experiments. **P<0.01, ***P<0.001, and ****P<0.0001, two-way ANOVA with post-test analysis.

FIGS. 4A-4O present data that show Myeloid lineage-specific deletion of Jag1 confers protection against UFP-induced exacerbation of allergic airway inflammation. FIG. 4A, Representative PAS-stained sections of lung isolated from Il4ra^{R576} or Il4ra^{R576}Lyz2^{Cre}Jag1^{Δ/Δ} mice in PBS, OVA or OVA+UFP groups (200× magnification). FIG. 4B, Inflammation scores in the lung tissues isolated from the mouse groups described in FIG. 4A. FIG. 4C-4G Airway hyper-responsiveness in response to methacholine (FIG. 4C), absolute numbers of eosinophils (FIG. 4D) and T cells (FIG. 4E) in the BAL fluids, total (FIG. 4F) and OVA-specific (FIG. 4G) levels in the serum of the mouse groups described in FIG. 4A. 4H-4K, Absolute numbers of lung Foxp3⁻CD4⁺ T cells secreting IL-4 (FIG. 4, H), IL13 (FIG. 4, I), IL-17 (FIG. 3, J) and IFN-γ (FIG. 4K) in the mouse groups described in FIG. 4, A. 4L-4O, Absolute numbers of lung Foxp3⁺CD4⁺ Treg cells secreting IL-4 (FIG. 4L), IL13 (FIG. 4M), IL-17 (FIG. 4N) and IFN-γ (FIG. 3O) in the mouse groups described in panel FIG. 4A. Results are representative of 2 independent experiments. N=5 mice/group. *p<0.05, **<0.01, ***<0.001, ****<0.0001 by two-way ANOVA with post test analysis.

FIGS. 5A-5O present data that show Jag1-sufficient AM rescue UFP-mediated allergic airway inflammation in Il4ra^{R576}Lyz2^{Cre}Jag1^{Δ/Δ} mice. FIG. 5A, Representative PAS-stained sections of lung tissues of Il4ra^{R576}Lyz2^{Cre}Jag1^{Δ/Δ} mice supplemented intra-tracheally with AM isolated from Il4ra^{R576} or Il4ra^{R576}Lyz2^{Cre}Jag1^{Δ/Δ} mice that were either sham treated (PBS) or loaded with OVA323-339 peptide (OVA) alone or together with UFP (OVA+UFP). FIG. 5B, Inflammation scores of lung tissues of mice described in FIG. 5A. C-G, Airway hyper-responsiveness (FIG. 5C), numbers of eosinophils (FIG. 5D) and T cells (FIG. 5E) in the BAL fluids, total (FIG. 5F) and OVA-specific (FIG. 5G) levels in the sera of mice described in FIG. 5A. 5H-5K, Numbers of lung Foxp3⁻CD4⁺ T cells secreting IL-4 (FIG. 5H), IL13 (FIG. 5I), IL-17 (FIG. 5J) and IFN-γ (FIG. 5K) in the BAL fluids of mice described in FIG. 5A. 5L-5O, Numbers of lung Foxp3⁺CD4⁺ Treg cells secreting IL-4 (FIG. 5L), IL13 (FIG. 5M), IL-17 (FIG. 5M) and IFN-γ (FIG. 5O) in the BAL fluids of mice described in FIG. 5A. Results are representative of 3 independent experiments. N=7-12 mice/group. *p<0.05, **<0.01, ***<0.001, ****<0.0001 by two-way ANOVA with Bonferroni post test analysis. n.s.: not significant.

FIGS. 6A-6B present data that show AM supports UFP-dependent upregulation of Th cell cytokine production by allergen-specific CD4⁺ T cells in a Notch4-dependent manner. FIG. 6A, Representative flow cytometric analysis of IL-4, IL-13 and IL-17 cytokine production by naive Il4ra^{R576}CD4⁺DO11.10⁺Rag2^{-/-} T cells co-cultured with FACS-purified AM isolated from l4ra^{R576} or Il4ra^{R576}Lyz2^{Cre}Jag1^{Δ/Δ} mice that have been pulsed with OVA323-339 peptide in the presence of UFP (10 μg/mL). Co-cultures were treated with either isotype control (Iso) Ab or an anti-Notch4 mAb, as indicated, and cytokine analysis was carried out on gated CD4⁺Foxp3⁻ T cell. FIG. 6B, Frequencies of T cells expressing the respective cytokine upon co-culture with AM that have been either sham treated (PBS) or pulsed with OVA323-339 peptide alone or in combination with UFP (10 μg/mL). Anti-Notch4 mAb or isotype control Ab were added as indicated. Results are representative of 3 independent experiments. *P<0.05, **P<0.01, ***P<0.001, and ****P<0.0001, two-way ANOVA with post-test analysis.

FIGS. 7A-7P present data that show UFP enhances allergic airway inflammation in a Notch4-dependent manner. FIG. 7A, Representative PAS staining of lung tissues isolated from Il4ra^{R576} mice sensitized and challenged with OVA alone, or together with UFP, in the presence of either isotype control (Iso) Ab or an anti-Notch4 mAb. FIG. 7B, Inflammation scores in lung tissues of the mouse groups described in in FIG. 7, A. 7C-7H Airway hyper-responsiveness in response to methacholine (FIG. 7, C), absolute numbers of eosinophils (FIG. 7D), T cells (FIG. 7E) and neutrophils (FIG. 7F) in the BAL fluids, total (FIG. 7G) and OVA-specific (FIG. 7H) levels in the serum of the mouse groups described in FIG. 7A. FIG. 7I-7L, Absolute numbers of lung Foxp3⁻CD4⁺ T cells secreting IL-4 (FIG. 7I), IL13 (FIG. 7J), IL-17 (FIG. 7K) and IFN-γ (FIG. 7L) in the mouse groups described in FIG. 7A. FIG. 7M-7P, Absolute numbers of lung Foxp3⁺CD4⁺ Treg cells secreting IL-4 (FIG. 7L), IL13 (FIG. 7M), IL-17 (FIG. 7N) and IFN-γ (FIG. 7O) in the mouse groups described in panel FIG. 7A. Results are representative of 2 independent experiments. N=5 mice/group. *p<0.05, **<0.01, ***<0.001, ****<0.0001 by two-way ANOVA with post test analysis.

FIG. 8A-8G present data that show disposition of fluorescent nanobeads (“Fluoresbrite YG”) in different lung cell subpopulations in mice subjected to sham (PBS), OVA or OVA+UFP-induced allergic airway inflammation. Mice were sensitized with OVA then challenged with PBS (sham treatment), OVA or OVA+UFP. Fluorescent nanobeads were introduced by intranasal instillation in all challenge groups. FIGS. 8A and 8B, Flow cytometric analysis of the uptake of fluorescent nanoparticles by CD45⁺ lung cell populations (Fig E1, A) and in lung macrophage populations segregated by the markers F4/80 and CD11c (Fig E1, A) followed by CD11b and CD11c (Fig E1, B), in mice subjected to allergic airway inflammation. FIG. 8C-8G, Distribution of nanobeads in CD45⁺F4/80⁻ cell fraction, sequentially segregated by the indicated markers and respective gates. Results are representative of 2 independent experiments. N=3 mice/group/experiment.

FIG. 9A-9D present data that show fluoresbrite YG nanobeads do not induce Jag1 expression in AM or influence allergic airway inflammation in mice. FIG. 9A, Flow cytometric analysis of Jag1 expression in cell-sorted AM cultured in vitro for 24 hr and treated with either PBS (sham treatment), Fluoresbrite YG nanobeads or UFP (at 10 μg/ml, respectively). FIG. 9B-9D, Disposition and effect of fluorescent nanobeads “Fluoresbrite YG” in the OVA model of allergic airway inflammation. Mice were sensitized with OVA then challenged with PBS (sham treatment), or OVA. Fluoresbrite YG nanobeads were introduced by intranasal instillation in a separate OVA-sensitized and challenged group. FIG. 9B, Flow cytometric analysis of the uptake of Fluoresbrite YG nanobeads by CD45⁺ lung cell populations and in lung macrophage populations segregated by the markers F4/80 and CD11c (followed by CD11b and CD11c, in mice subjected to allergic airway inflammation. FIG. 9C, 9D, Airway hyper-responsiveness (RI) and lung eosinophil and lymphocyte counts in the respective mouse groups. N=5 mice/group.

FIG. 10A-10C present data that show characterization of fluorescent nanoparticles-uptaking lung macrophages from mice subjected to OVA+UFP-induced allergic airway inflammation. FIGS. 10A and 10B, CD45⁺F4/80⁺CD11b^{Int}CD11c^{Hi} AM cells (Fig E2, A) and CD45⁺F4/80⁺CD11b^{Hi}CD11c^{Int} IM cells (FIG. 9B) were stained for the markers CD64, CD38, Egr2 and MHC class II I-A^{d}, as indicated. Results are representative of two independent experiments, N=3/group/experiment. FIG. 10C, Production of IL-6, IL-10, IL-12 p40 subunit, TNFα and CCL17 by cell-sorted AM treated in vitro for 48 hr with UFP at 10 μg/ml. N=5 independent cultures/cytokine assay. p<****<0.0001 by Student two tailed t test.

FIG. 11A-11B present data that show disposition of fluorescent microparticles in different lung cell subpopulations in mice following intranasal instillation under otherwise non-inflammatory conditions (no allergic sensitization). FIG. 11A, Flow cytometric analysis of the uptake of fluorescent microparticles by CD45⁺ lung cell populations and in lung macrophage populations segregated by the markers F4/80 and CD11c followed by CD11b and CD11c. FIG. 11B, Flow cytometric analysis of the uptake of microparticles in the CD45⁺F4/80⁻ cell fraction, sequentially segregated by the indicated markers and respective gates. Results are representative of 2 independent experiments. N=3 mice/group/experiment.

FIG. 12A-12B present data that show UFP upregulate Jag1 expression in BM-derived macrophages. FIG. 12A, Flow cytometric analysis of Jag1 expression in BM-derived macrophages prepared from either Il4ra^{R576} or Il4ra^{R576}Lyz2^{Cre}Ahr1^{Δ/Δ} mice and treated in vitro with either PBS, UFP (10 μg/ml), 6-FICZ (300 nM) or CB (10 μg/ml). FIG. 12B, Flow cytometric analysis of Jag1 expression in AM, IM and DC in lungs of Il4ra^{R576} or Il4ra^{R576}Lyz2^{Cre}Ahr1^{Δ/Δ} mice sensitized with either PBS or OVA and challenged with OVA or OVA+UFP.

FIG. 13A-13D present data that show AM support UFP-dependent upregulation of Th cell cytokine production by allergen-specific CD4⁺ T cells in a Jag1-dependent manner. FIG. 13A-13D, Representative flow cytometric analysis of IL-4 (FIG. 12A), IL-13 (FIG. 12B), IL-17 (FIG. 12C) and IFN-γ (FIG. 12D) cytokine production by naive Il4ra^{R576}CD4⁺DO11.10⁺Rag2^{-/-} T cells co-cultured with FACS-purified AM isolated from Il4ra^{R576} or Il4ra^{R576}Lyz2^{Cre}Jag1^{Δ/Δ} mice either sham-pulsed (PBS) or pulsed with OVA₃₂₃₋₃₃₉ peptide either alone (OVA) or in the presence of UFP (OVA+UFP).

FIG. 14A-14M present data that show myeloid lineage-specific deletion of Jag1 confers protection against the exacerbation by UFP of allergic airway inflammation induced by DM. FIG. 14A, Representative Periodic acid-Schiff (PAS)-stained sections of lung isolated from Il4ra^{R576} or Il4ra^{R576}Lyz2^{Cre}Jag1^{Δ/Δ} mice in PBS, DM or DM+UFP groups. FIG. 14B, Inflammation scores in the lung tissues isolated from the mouse groups described in Fig E6, A. FIG. 14C-14E Absolute numbers of eosinophils (Fig E6, C) and T cells (Fig E6, D) in the BAL fluids and serum total IgE concentrations (Fig E6, E) in the mouse groups described in FIG. 13A. FIG. 14F-14I, Absolute numbers of lung Foxp3⁻CD4⁺ T cells secreting IL-4 (FIG. 13F), IL13 (FIG. 13G, IL-17 (FIG. 13H) and IFN-γ (FIG. 13I) in the mouse groups described in FIG. 13A. FIG. 14J-14M, Absolute numbers of lung Foxp3⁺CD4⁺ Treg cells secreting IL-4 (FIG. 13J), IL13 (FIG. 13K), IL-17 (FIG. 13L) and IFN-γ (FIG. 13M) in the mouse groups described in panel FIG. 3A. Results are representative of 2 independent experiments. N=5 mice/group. *p<0.05, **<0.01, ***<0.001, ****<0.0001 by two-way ANOVA with post test analysis.

FIG. 15A-15N present data that show myeloid lineage-specific deletion of Ahr confers protection against UFP-induced exacerbation of allergic airway inflammation. FIG. 15A, Representative Periodic acid-Schiff (PAS)-stained sections of lung isolated from Il4ra^{R576} or Il4ra^{R576}Lyz2^{Cre}Ahr1^{Δ/Δ} mice in PBS, OVA or OVA+UFP groups. FIG. 15B, Inflammation scores in the lung tissues isolated from the mouse groups described in FIG. 14A. FIG. 15C-15F Absolute numbers of eosinophils (FIG. 14C) and T cells (FIG. 14D) in the BAL fluids and serum total (FIG. 14E) and OVA-specific (FIG. 14 F) IgE concentrations in the mouse groups described in FIG. 14 A. FIG. 15G-15J, Absolute numbers of lung Foxp3⁻CD4⁺ T cells secreting IL-4 (FIG. 14G), IL13 (FIG. 14H, IL-17 (FIG. 14I) and IFN-γ (FIG. 14J) in the mouse groups described in FIG. 14A. FIG. 15K-15N, Absolute numbers of lung Foxp3⁺CD4⁺ Treg cells secreting IL-4 (FIG. 14 K), IL13 (FIG. 14 L), IL-17 (FIG. 14 M) and IFN-γ (FIG. 14N) in the mouse groups described in panel FIG. 14A. Results are representative of 2 independent experiments. N=5 mice/group. *p<0.05, **<0.01, ***<0.001, ****<0.0001 by two-way ANOVA with post test analysis.

FIG. 16A-16F present data that show CD11c^{Cre}-mediated deletion of Jag1 does not protect against the exacerbation by UFP of allergic airway inflammation induced by DM. FIG. 16A, Representative Periodic acid-Schiff (PAS)-stained sections of lung isolated from Il4ra^{R576} or Il4ra^{R576} CD11c^{Cre} Jag1^{Δ/Δ} mice in PBS, OVA or OVA+UFP groups. FIG. 16B, Inflammation scores in the lung tissues isolated from the mouse groups described in FIG. 15 A. FIG. 16C-16F Absolute numbers of eosinophils (FIG. 15 C) and T cells (FIG. 15 D) in the BAL fluids and serum total and OVA-specific IgE concentrations (FIGS. 15 E and 15F) in the mouse groups described in FIG. 15 A. Results are representative of 2 independent experiments. N=5 mice/group. *p<0.05, **<0.01, ***<0.001, ****<0.0001 by two-way ANOVA with post test analysis.

FIG. 17A-17L present data that show jag1-sufficient IM and DC fail to rescue UFP-mediated allergic airway inflammation in Il4ra^{R576}Lyz2^{Cre}Jag1^{Δ/Δ} mice. FIG. 17A-17F, IM transfer; FIG. 17G-17L, DC transfer. Airway hyper-responsiveness in response to methacholine (FIGS. 16 A and 16G), absolute numbers of eosinophils (FIGS. 16B and 16H), OVA-specific serum IgE antibody concentrations (FIGS. 5C and 5I), and lung tissue CD4⁺ T cells (FIGS. 5D and 5J), absolute numbers of lung CD4⁺Foxp3⁻ T cells secreting IL13 and IL-17 (FIGS. 5E and 5K), Absolute numbers of lung Foxp3⁺CD4⁺ Treg cells secreting IL13 and IL-17 (FIGS. 16F and 16L). N=4 mice/group. *p<0.05, **<0.01, ***<0.001, ****<0.0001 by two-way ANOVA with Bonferroni post test analysis.

FIG. 18 present data that show flow cytometric analysis of Notch receptor expression on CD4⁺ T cells in allergic airway inflammation. Left panels: Representative flow cytometric analysis of Notch1-4 staining on CD4⁺ T cells in lungs of mice sensitized with PBS (sham) or OVA then challenged with OVA, or sensitized with OVA and challenged with OVA and UFP (OVA+UFP). Right panels: Frequencies of CD4⁺ T cells expressing the respective Notch receptor. Results are representative of 2 independent experiments; n=4 mice/group. ***P<0.001, and ****P<0.0001, one-way ANOVA with post-test analysis.

FIG. 19A-19D present data that show effect of neutralizing anti-Notch1-4 mAb treatment on the upregulation of Th cell cytokine production by allergen-specific CD4Foxp3⁻ T cells induced by OVA₃₂₃₋₃₃₉+UFP-treated AM. FIG. 19A-19D, Bar graph distribution of IL-4 (FIG. 16 A), IL-13 (FIG. 16 B), IL-17 (FIG. 16 C) and IFN-γ (FIG. 16 D) cytokine production by naive Il4ra^{R576}CD4⁺DO11.10⁺Rag2^{-/-} T cells co-cultured with FACS-purified AM isolated from l4ra^{R576} or Il4ra^{R576}Lyz2^{Cre}Jag1^{Δ/Δ} mice either sham-pulsed (PBS) or pulsed with OVA323-339 peptide either alone (OVA) or in the presence of UFP (OVA+UFP), as indicated. Control or anti-Notch Ab were added as indicated. Results represent means+S.E.M of 6 replicates derived from two independent experiments. **<0.01, ***<0.001, ****<0.0001 by one-way ANOVA with post test analysis.

FIG. 20A-20B present data that show effect of neutralizing anti-Notch 4 mAb treatment on the upregulation of Th cell cytokine production by allergen-specific CD4Foxp3⁺ Treg cells induced upon co-culture with allergen+UFP-pulsed AM. FIG. 20A, Representative flow cytometric analysis of IL-4, IL-13 and IL-17 cytokine production by CD4⁺Foxp3⁺ Treg cells induced upon the co-culture of Il4ra^{R576}CD4⁺DO11.10⁺Rag2^{-/-} naïve T cells with FACS-purified AM isolated from l4ra^{R576} or Il4ra^{R576}Lyz2^{Cre}Jag1^{Δ/Δ} mice and pulsed with OVA₃₂₃₋₃₃₉ and UFP (10 μg/mL). Co-cultures were treated with either isotype control Ab (Iso Ab) or an anti-Notch4 mAb, as indicated, and cytokine analysis was carried out on gated CD4⁺Foxp3⁺ T cell. FIG. 20B, Frequencies of Treg cells expressing the respective cytokine upon co-culture with AM that have been either sham treated (PBS) or pulsed with OVA₃₂₃₋₃₃₉ peptide alone or in combination with UFP (10 μg/mL). Anti-Notch4 mAb or isotype control Ab were added as indicated. Results are representative of 3 independent experiments. *P<0.05, **P<0.01, ***P<0.001, and ****P<0.0001, two-way ANOVA with post-test analysis.

FIG. 21A-21C present data that show inhibition of Notch target gene expression in T cells stimulated with allergen+UFP by treatment with neutralizing anti-Notch 4 mAb. DO11.10⁺ T cells were either sham treated (PBS+ control Ab; white bars) or co-cultured with AM pulsed with OVA₃₂₃₋₃₃₉ peptide+UFP in the presence of either control Ab (black bars) or anti-Notch4 mAb (grey bars), then assayed by real time PCR for the expression of transcripts of the respective target gene. FIG. 21A, Hes1 expression. FIG. 21B, Hey1 expression. FIG. 21C, Nrarp expression N=4 cultures/group. *<0.05, ***<0.001 by one-way ANOVA with post test analysis.

FIGS. 22A-22H present data that show interruption of Notch signaling in Treg cells protects against allergic airway inflammation. FIG. 22A. PAS staining of lung sections of Foxp3^{GFPCre} (control) mice versus mice with the Treg cell-specific deletion of the indicated Notch component. FIG. 22B. AHR in the indicated mouse strains immunized/challenged with PBS/OVA or OVA/OVA. 22C. Total and OVA-specific IgE. FIG. 22D. Lung tissue eosinophils and neutrophils. 22E-22H: frequencies of Foxp3⁺ Treg cells (FIG. 22E), and IL-4⁺ and IL-13⁺ (FIG. 22F), IL-17⁺ (FIG. 22G) and IFN-γ⁺ CD4⁺ T cells in lung tissues of the respective mouse groups. N=6-12 replicates/group derived from 3 experiments. *p<0.05, **<0.01, ***<0.001, ****<0.0001 by 2 way ANOVA and Bonferroni post-test analysis.

FIG. 23 presents data that show incapacitation of Notch signaling in Treg cells protects against UFP-induced exacerbation of allergic airway inflammation. AHR in the indicated mouse groups sensitized and challenged with PBS-OVA, OVA-OVA or OVA-OVA+UFP, as specified. Results representative of 5-6 mice from two independent experiments. *p<0.05, **<0.01, ***<0.001, by 2 way ANOVA and Bonferroni post-test analysis

FIGS. 24A-24C present data that show Notch4 expression is sharply upregulated on lung Treg cells by allergens and UFP in allergic airway inflammation. FIG. 24A. Notch1-4 mRNA expression in lung CD4⁺ Tconv and Treg cells. CD4⁺Foxp3⁺(YFP⁺) and CD4⁺Foxp3⁻ (YFP⁻) cells were isolated by cell sorting from the lungs of Foxp3^{YFPCre} mice that were either sensitized with either PBS (sham) or OVA then challenged with 1% nebulized OVA once daily for 3 days either alone or together with intranasal instillation of UFP (10 μg/d×3). The expression of Notch1/4 mRNA transcripts was analyzed by real time PCR and normalized for actin transcripts. FIG. 24B. present data that show Flow cytometric analysis of Notch4 expression on lung Treg and Tconv cells (upper and lower rows, respectively) in mice subjected to sham- (PBS), OVA- or OVA+UFP-induced allergic airway inflammation. FIG. 24C. Graphic display of Notch4 expression in the different cell groups shown in FIG. 24B. ***P<0.001 and ****P<0.0001 by One way ANOVA with post test analysis.

FIGS. 25A-25H present data that show Treg cell-specific deletion of Notch4 confers protection against allergen and UFP-induced allergic airway inflammation. 25A, Representative PAS-stained sections of lung isolated from Foxp3^{YFPCre} or Foxp3^{YFPCre}Notch4^{Δ/Δ} mice in PBS, OVA or OVA+UFP groups (200× magnification). FIG. 25B, Inflammation scores in the lung tissues isolated from the mouse groups described in panel A. 25C-25G Airway hyper-responsiveness in response to methacholine (FIG. 25C), absolute numbers of eosinophils (FIG. 25D) serum OVA-specific IgE levels (FIG. 25E), absolute numbers of lung Foxp3⁻CD4⁺ T cells (FIG. 25F), lung Foxp3⁻CD4⁺ T cells secreting IL-13 (FIG. 25G and IL-17 (FIG. 25H), Results are representative of 2 independent experiments. N=5 mice/group. p**<0.01, ***<0.001, ****<0.0001 by two-way ANOVA with post test analysis.

FIGS. 26A-26D present data showing that Notch4 expression is increased in circulating T regulatory cells of asthmatic subjects is a biomarker of disease severity. FIG. 26A-26B. Notch4 expression in circulating CD4+Foxp3+T regulatory cells and CD4+Foxp3− T effector cells in control subjects and in subjects with mild persistent, moderate persistent and severe asthma. FIGS. 26C-26D. Cell frequencies of Notch4 expressing cells and the mean fluorescence intensity (MFI) of Notch4 expression in CD4+Foxp3+T regulatory cells and CD4+Foxp3− T effector cells in the respective subject group. N=6-10 subjects/group. *p<0.05, **p<0.01, ***p<0.001, ****p<0.0001 by one way ANOVA with post-test analysis.

### DETAILED DESCRIPTION

The invention described herein is based in part on the discovery that activation of the Notch4 signaling pathway was sufficient to induce cells to differentiate into asthma causing T helper (Th) cells. Using an in vitro cell culture assays using lung-derived antigen presenting cells and allergen-specific T cells, and mouse models of allergic airway inflammation it was found that lung alveolar macrophage (AM) as the key cellular target of UFP in promoting airway inflammation.

Aryl hydrocarbon receptor (AhR)-dependent induction of Jagged 1 (Jag1) expression in AM was necessary and sufficient for the augmentation of allergic airway inflammation by UFP. UFP promoted Th2 and Th17 cell differentiation of allergen-specific T cells in a Jag1- and Notch4-dependent manner. Data presented herein specifically show that treatment of mice with an anti-Notch 4 antibody abrogated the exacerbation of allergic airway inflammation induced by UFP by preventing the differentiation of Th cells. It is specifically contemplated herein that an anti-Notch4 antibody will be useful in treating and/or preventing diseases caused by exposure to environmental irritants (e.g., ultra fine particles), such as asthma and allergic diseases.

Additionally, disclosed herein are methods for 1) identifying a subject at risk of having asthma or an allergic disease, and 2) measuring the efficacy of an asthma or allergic disease. These methods are based in part on the discovery that an abundance of Notch4 expression and/or activity corresponds with the prevalence of the disease or disorder. The use of Notch4 expression or activity as an indicator for asthma or an allergic disease in various methods is specifically contemplated herein.

Notch4

The Notch signaling pathway is an evolutionarily conserved intercellular signaling pathway that regulates interactions between physically adjacent cells. Notch signaling regulates multiple cell fate decisions; each Notch family member plays a role in a variety of developmental processes. In mammals, the Notch family is composed of four Notch receptors (Notch1-Notch4) and five ligands [Delta-like ligand 1 (DLL1), DLL3, DLL4, Jagged(Jag)1 and Jag2]. Upon binding to Jagged or Delta-like ligands on an adjacent cell, two sequential proteolytic events release the intracellular domain of Notch (NICD) allowing its translocation to the nucleus. There the NICD converts the DNA binding factor RBP-J from a transcriptional repressor to a transcriptional activator through MAML1-MAML3 binding¹.

The notch protein is cleaved in the trans-Golgi network, and then presented on the cell surface as a heterodimer. The protein functions as a receptor for membrane bound ligands, and may play a role in vascular, renal, and hepatic development.

SEQ ID NO: 1 contains a nucleic acid sequence that encodes Notch 4.

(SEQ ID NO: 1)
a tgcagccccc ttcactgctg ctgctgctgc tgctgctgct
gctgctatgt gtctcagtgg tcagacccag agggctgctg tgtgggagtt tcccagaacc
ctgtgccaat ggaggcacct gcctgagcct gtctctggga caagggacct gccagtgtgc
ccctggcttc ctgggtgaga cgtgccagtt tcctgacccc tgccagaacg cccagctctg
ccaaaatgga ggcagctgcc aagccctgct tcccgctccc ctagggctcc ccagctctcc
ctctccattg acacccagct tcttgtgcac ttgcctccct ggcttcactg gtgagagatg
ccaggccaag cttgaagacc cttgtcctcc ctccttctgt tccaaaaggg gccgctgcca
catccaggcc tcgggccgcc cacagtgctc ctgcatgcct ggatggacag gtgagcagtg
ccagcttcgg gacttctgtt cagccaaccc atgtgttaat ggaggggtgt gtctggccac
atacccccag atccagtgcc actgcccacc gggcttcgag ggccatgcct gtgaacgtga
tgtcaacgag tgcttccagg acccaggacc ctgccccaaa ggcacctcct gccataacac
cctgggctcc ttccagtgcc tctgccctgt ggggcaggag ggtccacgtt gtgagctgcg
ggcaggaccc tgccctccta ggggctgttc gaatgggggc acctgccagc tgatgccaga
gaaagactcc acctttcacc tctgcctctg tcccccaggt ttcataggcc cagactgtga
ggtgaatcca gacaactgtg tcagccacca gtgtcagaat gggggcactt gccaggatgg
gctggacacc tacacctgcc tctgcccaga aacctggaca ggctgggact gctccgaaga
tgtggatgag tgtgagaccc agggtccccc tcactgcaga aacgggggca cctgccagaa
ctctgctggt agctttcact gcgtgtgtgt gagtggctgg ggcggcacaa gctgtgagga
gaacctggat gactgtattg ctgccacctg tgccccggga tccacctgca ttgaccgggt
gggctctttc tcctgcctct gcccacctgg acgcacagga ctcctgtgcc acttggaaga
catgtgtctg agccagccgt gccatgggga tgcccaatgc agcaccaacc ccctcacagg
ctccacactc tgcctgtgtc agcctggcta ttcggggccc acctgccacc aggacctgga
cgagtgtctg atggcccagc aaggcccaag tccctgtgaa catggcggtt cctgcctcaa
cactcctggc tccttcaact gcctctgtcc acctggctac acaggctccc gttgtgaggc
tgatcacaat gagtgcctct cccagccctg ccacccagga agcacctgtc tggacctact
tgccaccttc cactgcctct gcccgccagg cttagaaggg cagctctgtg aggtggagac
caacgagtgt gcctcagctc cctgcctgaa ccacgcggat tgccatgacc tgctcaacgg
cttccagtgc atctgcctgc ctggattctc cggcacccga tgtgaggagg atatcgatga
gtgcagaagc tctccctgtg ccaatggtgg gcagtgccag gaccagcctg gagccttcca
ctgcaagtgt ctcccaggct ttgaagggcc acgctgtcaa acagaggtgg atgagtgcct
gagtgaccca tgtcccgttg gagccagctg ccttgatctt ccaggagcct tcttttgcct
ctgcccctct ggtttcacag gccagctctg tgaggttccc ctgtgtgctc ccaacctgtg
ccagcccaag cagatatgta aggaccagaa agacaaggcc aactgcctct gtcctgatgg
aagccctggc tgtgccccac ctgaggacaa ctgcacctgc caccacgggc actgccagag
atcctcatgt gtgtgtgacg tgggttggac ggggccagag tgtgaggcag agctaggggg
ctgcatctct gcaccctgtg cccatggggg gacctgctac ccccagccct ctggctacaa
ctgcacctgc cctacaggct acacaggacc cacctgtagt gaggagatga cagcttgtca
ctcagggcca tgtctcaatg gcggctcctg caaccctagc cctggaggct actactgcac
ctgccctcca agccacacag ggccccagtg ccaaaccagc actgactact gtgtgtctgc
cccgtgcttc aatgggggta cctgtgtgaa caggcctggc accttctcct gcctctgtgc
catgggcttc cagggcccgc gctgtgaggg aaagctccgc cccagctgtg cagacagccc
ctgtaggaat agggcaacct gccaggacag ccctcagggt ccccgctgcc tctgccccac
tggctacacc ggaggcagct gccagactct gatggactta tgtgcccaga agccctgccc
acgcaattcc cactgcctcc agactgggcc ctccttccac tgcttgtgcc tccagggatg
gaccgggcct ctctgcaacc ttccactgtc ctcctgccag aaggctgcac tgagccaagg
catagacgtc tcttcccttt gccacaatgg aggcctctgt gtcgacagcg gcccctccta
tttctgccac tgcccccctg gattccaagg cagcctgtgc caggatcacg tgaacccatg
tgagtccagg ccttgccaga acggggccac ctgcatggcc cagcccagtg ggtatctctg
ccagtgtgcc ccaggctacg atggacagaa ctgctcaaag gaactcgatg cttgtcagtc
ccaaccctgt cacaaccatg gaacctgtac tcccaaacct ggaggattcc actgtgcctg
ccctccaggc tttgtggggc tacgctgtga gggagacgtg gacgagtgtc tggaccagcc
ctgccacccc acaggcactg cagcctgcca ctctctggcc aatgccttct actgccagtg
tctgcctgga cacacaggcc agtggtgtga ggtggagata gacccctgcc acagccaacc
ctgctttcat ggagggacct gtgaggccac agcaggatca cccctgggtt tcatctgcca
ctgccccaag ggttttgaag gccccacctg cagccacagg gccccttcct gcggcttcca
tcactgccac cacggaggcc tgtgtctgcc ctcccctaag ccaggcttcc caccacgctg
tgcctgcctc agtggctatg ggggtcctga ctgcctgacc ccaccagctc ctaaaggctg
tggccctccc tccccatgcc tatacaatgg cagctgctca gagaccacgg gcttgggggg
cccaggcttt cgatgctcct gccctcacag ctctccaggg ccccggtgtc agaaacccgg
agccaagggg tgtgagggca gaagtggaga tggggcctgc gatgctggct gcagtggccc
gggaggaaac tgggatggag gggactgctc tctgggagtc ccagacccct ggaagggctg
cccctcccac tctcggtgct ggcttctctt ccgggacggg cagtgccacc cacagtgtga
ctctgaagag tgtctgtttg atggctacga ctgtgagacc cctccagcct gcactccagc
ctatgaccag tactgccatg atcacttcca caacgggcac tgtgagaaag gctgcaacac
tgcagagtgt ggctgggatg gaggtgactg caggcctgaa gatggggacc cagagtgggg
gccctccctg gccctgctgg tggtactgag ccccccagcc ctagaccagc agctgtttgc
cctggcccgg gtgctgtccc tgactctgag ggtaggactc tgggtaagga aggatcgtga
tggcagggac atggtgtacc cctatcctgg ggcccgggct gaagaaaagc taggaggaac
tcgggacccc acctatcagg agagagcagc ccctcaaacg cagcccctgg gcaaggagac
cgactccctc agtgctgggt ttgtggtggt catgggtgtg gatttgtccc gctgtggccc
tgaccacccg gcatcccgct gtccctggga ccctgggctt ctactccgct tccttgctgc
gatggctgca gtgggagccc tggagcccct gctgcctgga ccactgctgg ctgtccaccc
tcatgcaggg accgcacccc ctgccaacca gcttccctgg cctgtgctgt gctccccagt
ggccggggtg attctcctgg ccctaggggc tcttctcgtc ctccagctca tccggcgtcg
acgccgagag catggagctc tctggctgcc ccctggtttc actcgacggc ctcggactca
gtcagctccc caccgacgcc ggcccccact aggcgaggac agcattggtc tcaaggcact
gaagccaaag gcagaagttg atgaggatgg agttgtgatg tgctcaggcc ctgaggaggg
agaggaggtg ggccaggctg aagaaacagg cccaccctcc acgtgccagc tctggtctct
gagtggtggc tgtggggcgc tccctcaggc agccatgcta actcctcccc aggaatctga
gatggaagcc cctgacctgg acacccgtgg acctgatggg gtgacacccc tgatgtcagc
agtttgctgt ggggaagtac agtccgggac cttccaaggg gcatggttgg gatgtcctga
gccctgggaa cctctgctgg atggaggggc ctgtccccag gctcacaccg tgggcactgg
ggagaccccc ctgcacctgg ctgcccgatt ctcccggcca accgctgccc gccgcctcct
tgaggctgga gccaacccca accagccaga ccgggcaggg cgcacacccc ttcatgctgc
tgtggctgct gatgctcggg aggtctgcca gcttctgctc cgtagcagac aaactgcagt
ggacgctcgc acagaggacg ggaccacacc cttgatgctg gctgccaggc tggcggtgga
agacctggtt gaagaactga ttgcagccca agcagacgtg ggggccagag ataaatgggg
gaaaactgcg ctgcactggg ctgctgccgt gaacaacgcc cgagccgccc gctcgcttct
ccaggccgga gccgataaag atgcccagga caacagggag cagacgccgc tattcctggc
ggcgcgggaa ggagcggtgg aagtagccca gctactgctg gggctggggg cagcccgaga
gctgcgggac caggctgggc tagcgccggc ggacgtcgct caccaacgta accactggga
tctgctgacg ctgctggaag gggctgggcc accagaggcc cgtcacaaag ccacgccggg
ccgcgaggct gggcccttcc cgcgcgcacg gacggtgtca gtaagcgtgc ccccgcatgg
gggcggggct ctgccgcgct gccggacgct gtcagccgga gcaggccctc gtgggggcgg
agcttgtctg caggctcgga cttggtccgt agacttggct gcgcgggggg gcggggccta
ttctcattgc cggagcctct cgggagtagg agcaggagga ggcccgaccc ctcgcggccg
taggttttct gcaggcatgc gcgggcctcg gcccaaccct gcgataatgc gaggaagata
cggagtggct gccgggcgcg gaggcagggt ctcaacggat gactggccct gtgattgggt
ggccctggga gcttgcggtt ctgcctccaa cattccgatc ccgcctcctt g

Treating or Preventing asthma or an allergic disease

One aspect of the invention is a method of treating asthma or an allergic disease by administering to a subject having asthma or an allergic disease an agent that inhibits Notch4. Another aspect provides a method of treating asthma or an allergic disease by identifying a subject having asthma or an allergic disease, and administering to a subject having asthma or an allergic disease an agent that inhibits Notch4.

As used herein, an “asthma” refers to a disease characterized by inflammation in the airways of the lungs, reversible airways obstructions, bronchospasms, wheezing, coughing, tightness of the chest, and shortness of breath. Asthma is thought to be caused by environmental and genetic factors, include, but not limited to exposure to air pollutants and allergens, aspirin and beta blockers, and a family history of asthma.

Asthma is classified by the frequency of symptoms, the severity of symptoms, forced expiratory volume in one second (FEV1), and peak expiratory flow rate. Asthma can further be classified based on the subject's response to a medication, e.g., atopic or non-atopic, wherein atropic refers to a predisposition towards developing a type 1 hypersensitivity.

In various embodiments, the asthma is allergic asthma (e.g., induced by exposure to allergens), asthma without allergies (e.g., induced by an upper respiratory infection, such as a cold, flu, or rhinovirus), aspirin exacerbated respiratory disease (e.g., induced by the intake of aspirin), exercised-induced asthma, cough variant (e.g., characterized by a dry, hacking cough), or occupational asthma (e.g., induced by an irritant a subject is exposed to on a job, for example, a fire fighter is exposed to smoke, and can experience smoke-inhalation, while performing their job). A skilled clinician can identify a type of asthma a subject has, or is at risk of having (e.g., a fire fighter would be at risk of having occupational asthma), using standard techniques.

As used herein, an “allergic disease” is a disease that is characterized by an immune system response to an otherwise harmless substance in the environment. For example, when a subject who has an allergic disease is exposed to common environmental substances the subject's B lymphocytes produce specific antibodies against that substance, resulting in an immune response. Exemplary substances that, e.g., can cause an allergic disease include dust mites, pollen (e.g., from plants, trees, flowers, or grass), animal dander (e.g., from domestic or farm animals), mold, food (e.g., tree nuts, peanuts, shellfish, fish, milk, eggs, or wheat), and latex. A child whose parent, or parents, have allergies are at an increased risk of developing an allergic disease. The specific cause of an allergic diseases (e.g., what the allergen is) can be identified by a skilled clinician using common techniques, e.g., skin prick tests and radioallergosorbent tests.

In one embodiment, the allergic disease is allergic rhinitis, sinusitis, otitis media, atopic dermatitis (e.g., eczema), urticaria, angioedema, and anaphylaxis.

A subject can be identified as having, e.g., asthma or an allergic reaction, by a skilled clinician. Diagnostic tests useful in identifying a subject having asthma or an allergic disease are known in the art, and further described herein below.

Another aspect of the invention is a method of preventing asthma or an allergic disease by administering to a subject who is at risk of developing asthma or an allergic disease an agent that inhibits Notch4. In one embodiment, the method further comprises identifying a subject at risk of developing asthma or an allergic reaction prior to administering the agent.

As used herein a subject “at risk of having asthma” refers to a subject who is in contact, or potentially in contact, with known asthma triggers (e.g., factors that can result in the onset of asthma). Non-limiting factors that can, e.g., trigger the onset of asthma or allergic disease, include airborne substances, (e.g., pollen, dust mites, mold spores, pet dander or particles of cockroach waste); respiratory infections, (e.g., the common cold); physical activity (e.g., can trigger exercised-induced asthma); cold air; air pollutants and irritants, (e.g., smoke and cigarette smoke); certain medications (e.g., blockers, aspirin, ibuprofen (Advil, Motrin IB, others) and naproxen (Aleve)); strong emotions or stress; sulfites and preservatives added food and/or beverages (e.g., found in shrimp, dried fruit, processed potatoes, beer, and wine); and gastroesophageal reflux disease (GERD). A subject is also considered at risk of asthma or an allergic disease if the subject has a family history of asthma or an allergic disease (e.g., if an immediate family member has had asthma or an allergic disease).

Agents

In one aspect, an agent that inhibits Notch4 is administered to a subject having, or at risk of having asthma or an allergic disease. In one embodiment, the agent that inhibits Notch4 is a small molecule, an antibody or antibody fragment, a peptide, an antisense oligonucleotide, a genome editing system, or an RNAi.

An agent is considered effective for inhibiting Notch4 if, for example, upon administration, it inhibits the presence, amount, activity and/or level of Notch4 in the cell.

In one embodiment, inhibiting Notch4 inhibits the differentiation of a Notch4-expressing Treg cell into a disease-promoting Th cell.

An agent can inhibit e.g., the transcription, or the translation of Notch4 in the cell. An agent can inhibit the activity or alter the activity (e.g., such that the activity no longer occurs, or occurs at a reduced rate) of Notch4 in the cell (e.g., Notch4's expression).

In one embodiment, an agent that inhibits Notch4 promotes programmed cell death, e.g., kill, the cell that expresses Notch4, for example, a T reg cell. To determine is an agent is effective at inhibiting Notch4, mRNA and protein levels of a given target (e.g., Notch4) can be assessed using RT-PCR and western-blotting, respectively. Biological assays that detect the activity of Notch4 (e.g., Notch reporters that measure the binding of the Notch receptor and ligand) can be used to assess if programmed cell death has occurred. Alternatively, immunofluorescence detection using antibodies specific to Notch4 in combination with cell death markers (e.g., Caspase) can be used to determine if cell death has occurred following administration of an agent.

In one embodiment, an agent that inhibits the level and/or activity of Notch4 by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the level and/or activity of Notch4 prior to administration of the agent, or the level and/or activity of Notch4 in a population of cells that was not in contact with the agent.

The agent may function directly in the form in which it is administered. Alternatively, the agent can be modified or utilized intracellularly to produce something which inhibits Notch4, such as introduction of a nucleic acid sequence into the cell and its transcription resulting in the production of the nucleic acid and/or protein inhibitor of Notch4. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits Notch4. Methods for screening small molecules are known in the art and can be used to identify a small molecule that is efficient at, for example, inducing cell death of pathogenic CD4 cells, given the desired target (e.g., Notch4).

In various embodiments, the agent that inhibits Notch4 is an antibody or antigen-binding fragment thereof, or an antibody reagent that is specific for Notch4. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the agent that inhibits Notch4 is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use.

In one embodiment, the antibody or antibody reagent binds to an amino acid sequence that corresponds to the amino acid sequence encoding Notch4 (SEQ ID NO: 2).

(SEQ ID NO: 2)
MQPPSLLLLLLLLLLLCVSVVRPRGLLCGSFPEPCANGGTCLSLSLGQGT
CQCAPGFLGETCQFPDPCQNAQLCQNGGSCQALLPAPLGLPSSPSPLTPS
FLCTCLPGFTGERCQAKLEDPCPPSFCSKRGRCHIQASGRPQCSCMPGWT
GEQCQLRDFCSANPCVNGGVCLATYPQIQCHCPPGFEGHACERDVNECFQ
DPGPCPKGTSCHNTLGSFQCLCPVGQEGPRCELRAGPCPPRGCSNGGTCQ
LMPEKDSTFHLCLCPPGFIGPDCEVNPDNCVSHQCQNGGTCQDGLDTYTC
LCPETWTGWDCSEDVDECETQGPPHCRNGGTCQNSAGSFHCVCVSGWGGT
SCEENLDDCIAATCAPGSTCIDRVGSFSCLCPPGRTGLLCHLEDMCLSQP
CHGDAQCSTNPLTGSTLCLCQPGYSGPTCHQDLDECLMAQQGPSPCEHGG
SCLNTPGSFNCLCPPGYTGSRCEADHNECLSQPCHPGSTCLDLLATFHCL
CPPGLEGQLCEVETNECASAPCLNHADCHDLLNGFQCICLPGFSGTRCEE
DIDECRSSPCANGGQCQDQPGAFHCKCLPGFEGPRCQTEVDECLSDPCPV
GASCLDLPGAFFCLCPSGFTGQLCEVPLCAPNLCQPKQICKDQKDKANCL
CPDGSPGCAPPEDNCTCHHGHCQRSSCVCDVGWTGPECEAELGGCISAPC
AHGGTCYPQPSGYNCTCPTGYTGPTCSEEMTACHSGPCLNGGSCNPSPGG
YYCTCPPSHTGPQCQTSTDYCVSAPCFNGGTCVNRPGTFSCLCAMGFQGP
RCEGKLRPSCADSPCRNRATCQDSPQGPRCLCPTGYTGGSCQTLMDLCAQ
KPCPRNSHCLQTGPSFHCLCLQGWTGPLCNLPLSSCQKAALSQGIDVSSL
CHNGGLCVDSGPSYFCHCPPGFQGSLCQDHVNPCESRPCQNGATCMAQPS
GYLCQCAPGYDGQNCSKELDACQSQPCHNHGTCTPKPGGFHCACPPGFVG
LRCEGDVDECLDQPCHPTGTAACHSLANAFYCQCLPGHTGQWCEVEIDPC
HSQPCFHGGTCEATAGSPLGFICHCPKGFEGPTCSHRAPSCGFHHCHHGG
LCLPSPKPGFPPRCACLSGYGGPDCLTPPAPKGCGPPSPCLYNGSCSETT
GLGGPGFRCSCPHSSPGPRCQKPGAKGCEGRSGDGACDAGCSGPGGNWDG
GDCSLGVPDPWKGCPSHSRCWLLFRDGQCHPQCDSEECLFDGYDCETPPA
CTPAYDQYCHDHFHNGHCEKGCNTAECGWDGGDCRPEDGDPEWGPSLALL
VVLSPPALDQQLFALARVLSLTLRVGLWVRKDRDGRDMVYPYPGARAEEK
LGGTRDPTYQERAAPQTQPLGKETDSLSAGFVVVMGVDLSRCGPDHPASR
CPWDPGLLLRFLAAMAAVGALEPLLPGPLLAVHPHAGTAPPANQLPWPVL
CSPVAGVILLALGALLVLQLIRRRRREHGALWLPPGFTRRPRTQSAPHRR
RPPLGEDSIGLKALKPKAEVDEDGVVMCSGPEEGEEVGQAEETGPPSTCQ
LWSLSGGCGALPQAAMLTPPQESEMEAPDLDTRGPDGVTPLMSAVCCGEV
QSGTFQGAWLGCPEPWEPLLDGGACPQAHTVGTGETPLHLAARFSRPTAA
RRLLEAGANPNQPDRAGRTPLHAAVAADAREVCQLLLRSRQTAVDARTED
GTTPLMLAARLAVEDLVEELIAAQADVGARDKWGKTALHWAAAVNNARAA
RSLLQAGADKDAQDNREQTPLFLAAREGAVEVAQLLLGLGAARELRDQAG
LAPADVAHQRNHWDLLTLLEGAGPPEARHKATPGREAGPFPRARTVSVSV
PPHGGGALPRCRTLSAGAGPRGGGACLQARTWSVDLAARGGGAYSHCRSL
SGVGAGGGPTPRGRRESAGMRGPRPNPAIMRGRYGVAAGRGGRVSTDDWP
CDWVALGACGSASNIPIPPPCLTPSPERGSPQLDCGPPALQEMPINQGGE
GKK

In another embodiment, the anti-Notch4 antibody or antibody reagent binds to an amino acid sequence that comprises the sequence of SEQ ID NO: 2; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-Notch4 antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., Notch4, and inhibits the differentiation of a Notch4-expressing Treg cell into a disease-promoting Th cell.

In one embodiment, the agent that inhibits Notch4 is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., Notch4. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits Notch4 may comprise at least 5, at least 10, at least 15, at least 20, at least 25, at least 30, or more bases complementary to a portion of the coding sequence of the human Notch4 gene (e.g., SEQ ID NO: 1).

In one embodiment, Notch4 is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits Notch4 by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. Notch4. In some embodiments of any of the aspects, the agent is siRNA that inhibits Notch4. In some embodiments of any of the aspects, the agent is shRNA that inhibits Notch4.

One skilled in the art would be able to design siRNA, shRNA, or miRNA to target Notch4, e.g., using publically available design tools. siRNA, shRNA, or miRNA is commonly made using companies such as Dharmacon (Layfayette, Colo.) or Sigma Aldrich (St. Louis, Mo.).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits Notch4. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect WatsonCrick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., Notch4), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., Notch4, for its downregulation, for example by transfecting the siRNA, shRNA, or miRNA into cells and detecting the levels of a gene (e.g., Notch4) found within the cell via western-blotting.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., an Notch4 inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

Identifying a Subject at Risk of Having Asthma or an Allergic Disease

One aspect of the invention describe herein provides a method for identifying a subject at risk of having asthma or an allergic disease comprising, (a) obtaining a biological sample from the subject; (b) measuring the level of Notch4 in the sample, wherein the subject is at risk of having asthma or an allergic disease if the level of Notch is increased as compared to a reference level; and (c) administering an agent that inhibits Notch4 to a subject at risk.

In one embodiment, the level of Notch4 is increased at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or more as compared to the reference level, or at least 10%, at least 20%, at least 30%, at least 40%, at least 50%, at least 60%, at least 70%, at least 80%, at least 90%, or at least 99% or more as compared to the reference level. The reference level can be the level of Notch4 in a sample obtained from a healthy subject, e.g., a subject who is not at risk of asthma or an allergic reaction.

In one embodiment, the levels of Notch4 are measured in vitro, or ex vivo. The levels of Notch4 in the sample can be measured using standard techniques, e.g., FACS analysis, or immunofluorescence. Protein and mRNA levels of Notch4 can be assessed using western blotting or PCR-based assays, respectively, as described herein.

In one embodiment, the biological sample is a blood sample, a peripheral blood sample, a sputum sample, a lung tissue sample, a lung biopsy sample, or a bronchial lavage sample. In one embodiment, the biological sample is any sample that contains alveolar macrophages. In one embodiment, the biological sample is taken from a subject that has previously been diagnosed with asthma or an allergic disease. In one embodiment, the biological sample is taken from a subject that has previously been diagnosed with and treated for asthma or an allergic disease. In one embodiment, the biological sample is taken from a subject that has not been diagnosed with asthma or an allergic disease. Methods for collecting samples from a subject are known in art and can be performed by a skilled person.

Measuring Therapeutic Efficacy

One aspect of the invention provides a method of determining the efficacy of a therapeutic in the treatment of a subject diagnosed with asthma or an allergic disease comprising, (a) determining a first level of Notch4 expression or activity in a sample provided by the subject diagnosed with asthma or an allergic disease prior to the administration of a therapeutic; (b) determining a second level of Notch4 expression or activity in a sample provided by the patient after administration of the therapeutic; and (c) comparing said first and second levels of Notch4 expression or activity, wherein the therapeutic is considered effective if said second level of Notch4 expression or activity is lower than said first level, and wherein the therapeutic administered in (b) is ineffective if said second level of Notch4 expression is the same as or higher than said first level.

In one embodiment, a therapeutic is considered effective if the second level of Notch4 expression or activity is decreased at least 1%, at least 5%, at least 10%, at least 20%, at least about 30%, at least about 40%, at least about 50%, at least about 60%, at least about 70%, at least about 80%, at least about 90%, at least 99%, or 100% as compared to the first level of Notch4 expression or activity.

In one embodiment, the biological sample is a blood sample, a peripheral blood sample, a sputum sample, a lung tissue sample, a lung biopsy sample, or a bronchial lavage sample. In one embodiment, the biological sample is any sample that contains alveolar macrophages. Methods for collecting samples from a subject are known in art and can be performed by a skilled person.

In one embodiment, the biological sample is taken from a subject that has been diagnosed with asthma or an allergic disease, but has not been administered a therapeutic to treat asthma or an allergic disease. In one embodiment, the biological sample is taken from a subject that has been diagnosed with asthma or an allergic disease, but has been administered a therapeutic to treat asthma or an allergic disease.

In one embodiment, the therapeutic is an agent that inhibits Notch4. In another embodiment, the therapeutic is an anti-asthma or an anti-allergic disease therapeutic. Exemplary anti-asthma and an anti-allergic disease therapeutic are further described herein below.

Administration

In some embodiments, the methods described herein relate to treating a subject having or diagnosed as having an asthma or an allergic disease comprising administering an agent that inhibits Notch4 as described herein. Subjects having an asthma or an allergic disease can be identified by a physician using current methods of diagnosing a condition. Symptoms and/or complications of asthma or an allergic disease, which characterize these disease and aid in diagnosis are well known in the art and include but are not limited to, persistent cough, trouble breathing, wheezing, shortness of breath, and skin rash. Tests that may aid in a diagnosis of, e.g. asthma, include but are not limited methacholine challenge, nitric oxide test, allergy testing, and sputum eosinophils. A family history of, e.g., asthma, will also aid in determining if a subject is likely to have the condition or in making a diagnosis of asthma or an allergic disease.

The agents described herein (e.g., an agent that inhibits Notch4) can be administered to a subject having or diagnosed as having asthma or an allergic disease. In some embodiments, the methods described herein comprise administering an effective amount of an agent to a subject in order to alleviate at least one symptom of, e.g., asthma. As used herein, “alleviating at least one symptom of asthma or an allergic disease” is ameliorating any condition or symptom associated with, e.g., asthma (e.g., persistent cough, trouble breathing, wheezing, shortness of breath, and skin rash). As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. In one embodiment, the agent is administered systemically or locally (e.g., to the lungs). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner.

In one embodiment, the agent, or compositions comprising an agent is administered through inhalation.

The term “effective amount” as used herein refers to the amount of an agent (e.g., an agent that inhibits Notch4) can be administered to a subject having or diagnosed as having asthma or an allergic disease needed to alleviate at least one or more symptom of, e.g., asthma. The term “therapeutically effective amount” therefore refers to an amount of an agent that is sufficient to provide, e.g., a particular anti-asthma effect when administered to a typical subject. An effective amount as used herein, in various contexts, would also include an amount of an agent sufficient to delay the development of a symptom of, e.g., asthma, alter the course of a symptom of, e.g., asthma (e.g., slowing the progression of loss of lung function, inappropriate breathing, or wheezing), or reverse a symptom of, e.g., (e.g., improve lung function or breathing). Thus, it is not generally practicable to specify an exact “effective amount”. However, for any given case, an appropriate “effective amount” can be determined by one of ordinary skill in the art using only routine experimentation.

In one embodiment, the agent is administered continuously (e.g., at constant levels over a period of time). Continuous administration of an agent can be achieved, e.g., by epidermal patches, continuous release formulations, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Combinational Therapy

In one embodiment, the agent described herein is used as a monotherapy. In one embodiment, the agents described herein can be used in combination with other known agents and therapies for asthma or an allergic disease. Administered “in combination,” as used herein, means that two (or more) different treatments are delivered to the subject during the course of the subject's affliction with the disorder, e.g., the two or more treatments are delivered after the subject has been diagnosed with the disorder or disease (asthma or an allergic disease) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Exemplary therapeutics used to treat asthma include, but are not limited to, inhaled corticosteroids (e.g., fluticasone (Flonase, Flovent HFA), budesonide (Pulmicort Flexhaler, Rhinocort), flunisolide (Aerospan HFA), ciclesonide (Alvesco, Omnaris, Zetonna), beclomethasone (Qnasl, Qvar), mometasone (Asmanex) and leukotriene modifiers (e.g., montelukast (Singulair), zafirlukast (Accolate) and zileuton (Zyflo)); long-acting beta agonists (e.g., salmeterol (Serevent) and formoterol (Foradil, Perforomist)); combination inhalers (e.g., fluticasone-salmeterol (Advair Diskus), budesonide-formoterol (Symbicort) and formoterol-mometasone (Dulera)); theophylline (e.g., Theophylline (Theo-24, Elixophylline)); short-acting beta agonists (e.g., albuterol (ProAir HFA, Ventolin HFA, others) and levalbuterol (Xopenex)); ipratropium (e.g., Atrovent); and oral and intravenous corticosteroids.

Exemplary therapeutics used to treat an allergic disease include, but are not limited to, anti-inflammatory therapeutics (e.g., corticosteroids, glucocorticoids, or mineralcorticoids); antihistamines (e.g., Brompheniramine (Dimetane), Cetirizine (Zyrtec), Chlorpheniramine (Chlor-Trimeton), Clemastine (Tavist), Diphenhydramine (Benadryl), Fexofenadine (Allegra), or Loratadine (Alavert, Claritin)); and adrenaline.

When administered in combination, the agent and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of asthma or an allergic disease) is lower (e.g., at least 20%, at least 30%, at least 40%, or at least 50% lower) than the amount or dosage of each agent individually required to achieve the same therapeutic effect.

Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol Formulations

A composition comprising an agent that inhibits Notch4 can be administered directly to the airways of a subject in the form of an aerosol or by nebulization. For use as aerosols, an agent that inhibits Notch4 in solution or suspension may be packaged in a pressurized aerosol container together with suitable propellants, for example, hydrocarbon propellants like propane, butane, or isobutane with conventional adjuvants. An agent that inhibits Notch4 can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device.

As is well known, any suitable gas can be used to apply pressure during the nebulization, with preferred gases to date being those which are chemically inert to a modulator of an agent that inhibits Notch4. Exemplary gases including, but are not limited to, nitrogen, argon or helium can be used to high advantage.

In some embodiments, an agent that inhibits Notch4 can also be administered directly to the airways in the form of a dry powder. For use as a dry powder, a GHK tripeptide can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The correct dosage of the composition is delivered to the patient.

A dry powder inhaler (i.e. Turbuhaler (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of an agent that inhibits Notch4 thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

Controlled and Delayed Release Dosage Forms

In some embodiments of the aspects described herein, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

Efficacy

The efficacy of an agents described herein, e.g., for the treatment of an asthma or an allergic disease, can be determined by the skilled practitioner. However, a treatment is considered “effective treatment,” as the term is used herein, if one or more of the signs or symptoms of, e.g., asthma, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., decreased airway inflammation, increased lung function, restored normal breathing. Efficacy can also be measured by a failure of an individual to worsen as assessed by hospitalization, or need for medical interventions (i.e., progression of diminished lung function, complications with breathing, asthmatic attack frequencies). Methods of measuring these indicators are known to those of skill in the art and/or are described herein.

Efficacy can be assessed in animal models of a condition described herein, for example, a mouse model or an appropriate animal model of asthma or allergic disease, as the case may be. When using an experimental animal model, efficacy of treatment is evidenced when a statistically significant change in a marker is observed, e.g., decreased airway inflammation, increased lung function, restored normal breathing.

Efficacy of an agent that inhibits Notch4 can additionally be assessed using methods described herein.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

1) A method for treating asthma or an allergic disease, comprising administering to a subject having asthma or an allergic disease an effective amount of an agent that inhibits Notch4.
2) A method for treating asthma or an allergic disease, comprising:
a. identifying a subject having asthma or an allergic disease; and
b. administering to a subject having asthma or an allergic disease an effective amount of an agent that inhibits Notch4.
   3) The methods of paragraphs 1 and 2, wherein the asthma is selected from the list consisting of allergic asthma, asthma without allergies, aspirin exacerbated respiratory disease, exercise induced asthma, cough variant, and occupational asthma.
   4) The methods of paragraphs 1 and 2, wherein the allergic disease is selected from the list consisting of allergic rhinitis, sinusitis, otitis media, atopic dermatitis, urticaria, angioedema, and anaphylaxis.
   5) The method of paragraphs 1 and 2, wherein the agent that inhibits Notch4 is selected from the group consisting of a small molecule, an antibody, a peptide, a genome editing system, an antisense oligonucleotide, and an RNAi.
   6) The method of paragraph 5, wherein the antibody is a humanized antibody.
   7) The method of paragraph 5, wherein the RNAi is a microRNA, an siRNA, or a shRNA. 8) The method of paragraphs 1-7, wherein inhibiting Notch4 is inhibiting the expression level and/or activity of Notch4.
   9) The method of paragraph 8, wherein the expression level and/or activity of Notch4 is inhibited by at least 50%, at least 60%, at least 70%, at least 80%, at least 90%, or more as compared to an appropriate control.
   10) The method of paragraphs 1 or 2, wherein Notch4 is inhibited on T regulatory cells.
   11) The method of paragraphs 1 and 2, further comprising administering at least one additional anti-asthma therapeutic.
   12) The method of paragraphs 1 and 2, further comprising administering at least one additional anti-allergic disease therapeutic.
   13) A method for preventing asthma or an allergic disease, comprising administering to a subject at risk of having asthma or an allergic disease an agent that inhibits Notch4.
   14) The method of paragraph 13, further comprising, prior to administering, identifying a subject at risk of having asthma or an allergic disease.
   15) A composition for the treatment of asthma or an allergic disease, the composition comprising an agent that inhibits Notch4 and a pharmaceutically acceptable carrier.
   16) The composition of paragraph 15, wherein the agent that inhibits Notch4 is selected from the group consisting of a small molecule, an antibody, a peptide, a genome editing system, an antisense oligonucleotide, and an RNAi.
   17) The composition of paragraph 15, wherein the antibody is a humanized antibody.
   18) The composition of paragraph 15, wherein the RNAi is a microRNA, an siRNA, or a shRNA.
   19) The composition of paragraph 15, wherein the composition is formulated for inhaled administration.
   20) A method for treating a subject at risk of having asthma or an allergic disease, the method comprising,
a. Obtaining a biological sample from the subject;
b. measuring the level of Notch4 in a population of candidate cells;
   wherein the subject is at risk of having asthma or an allergic disease if the level of Notch is increased as compared to a reference level; and
c. administering an agent that inhibits Notch4 to a subject at risk.
   21) The method of paragraphs 20, wherein the level of Notch4 is increased by at least 2-fold, at least 3-fold, at least 4-fold, at least 5-fold, at least 6-fold, at least 7-fold, at least 8-fold, at least 9-fold, at least 10-fold, or more as compared to a reference level.
   22) A method of determining the efficacy of a therapeutic in the treatment of a subject diagnosed with asthma or an allergic disease, the method comprising:
a) determining a first level of Notch4 expression or activity in a sample provided by the subject diagnosed with asthma or an allergic disease prior to the administration of a therapeutic;
b) determining a second level of Notch4 expression or activity in a sample provided by the patient after administration of the therapeutic; and
c) comparing said first and second levels of Notch4 expression or activity, wherein the therapeutic is considered effective if said second level of Notch4 expression or activity is lower than said first level, and wherein the therapeutic administered in (b) is ineffective if said second level of Notch4 expression is the same as or higher than said first level.
   23) The method of paragraph 22, wherein the therapeutic is an agent that inhibits Notch4.

### EXAMPLES

### Example 1

Introduction

It is well appreciated that exposure to air pollution, especially particulate matter (PM) emitted by combustion sources, play an important role in the increased incidence and prevalence of asthma in recent decades¹⁻⁶. Among air pollutants, exposure to PM shows the strongest correlation with adverse respiratory health effects⁷⁻¹⁰. These particles have been shown to promote Th2 and Th17 cell responses and upregulate IgE production in the exposed host¹¹⁻¹⁵ Inhaled PM exhibit differential airway penetrance that stratifies according to size. Unlike coarse particles (CP; ≥2.5 μm in diameter) trapped in the nasopharyngeal region, fine particles (FP; ≤2.5 μm in diameter) and ultra-fine particles (UFP; ≤0.2 μm in diameter) are able to penetrate into the lower respiratory tract where they are taken up by antigen-presenting cells (APC) to mediate local and systemic inflammation¹⁶. PM modulation of APC function maybe particularly relevant to the adjuvant-like effect of PM in promoting immune responses to allergens^{13,17}. Recent studies have identified a key mechanism common to both UFP and FP by which they augment allergic responses, involving their induction of the Notch receptor ligand Jagged1 (Jag1) on APC¹⁵. This induction is mediated by the activation by PM-associated polycyclic aromatic hydrocarbons (PAH) of the aryl hydrocarbon receptor (AhR), which in turn mediates the transcriptional activation of Jag1. Jag1 engages Notch receptors on allergen-specific T cells, leading to their augmented differentiation into disease-promoting Th cells. These studies did not precisely identify the relevant APC species involved in this process, nor the target Notch receptor(s) mediating the response to PM-induced Jag1.

Lung macrophages have previously been implicated in the uptake of and response to PM¹⁸⁻²⁰. They include two major subsets: alveolar macrophages (AM), expressing high levels of the β2 integrin CD11c (CD11c^{hi}) and interstitial macrophages (IM) expressing intermediate levels of CD11c (CD11c^{int})^{21,22.} Studies have shown that both populations promote immune tolerance in the steady state by inducing naive T cell to Treg cell differentiation^{23,24}. However, inflammatory stimuli, including allergens and endotoxin, modulate the expression of co-stimulation molecules and alter the potency of lung macrophage as antigen presenting cells^{25,26}. In a similar vein, exposure of AM to PM alters their function, rendering them pro-inflammatory²⁷. In addition to targeting lung macrophages, PM have been shown to potentiate the antigen presenting function of lung dendritic cells (DC)²⁸. The relative contribution of the respective APC type to the allergic airway inflammatory response induced by PM remains to be fully elucidated.

To investigate mechanisms by which PM exposure may target lung APC to promote allergic diseases, a range of genetic, immunological and whole animal approaches was employed. Provided herein is evidence for a critical role for PM-mediated, AhR-dependent Jag1 induction in AM in promoting allergic airway inflammation by a process involving Notch4-dependent allergen-specific T helper cell differentiation.

Methods and Materials

Mice. Il4ra^{R576} and Foxp3^{EGFP} mice were previously described^{15,29,30} The following mice were obtained from the Jax Lab: BLAB/c (WT), Ahr^{fl/fl} (Ahr^{tm3.1Bra})³¹, Lyz2^{Cre} (CreB6.129P2-Lyz2^{tm1(cre)Ifo}/J) and CD11c^{Cre} (B6.Cg-Tg(Itgax-cre)1-1Reiz/J)³². DO11.10Rag2^{-/-} mice were obtained from Taconic farms. They were crossed with Il4ra^{R576} mice to generate DO11.10Rag2^{-/-}Il4ra^{R576}Foxp3^{EGFP} mice³⁰. Jag1^{fl/fl} mice were kindly provided by Dr. Freddy Radtke³³.

Particles. UFP (≤0.18 μm) were collected in an urban area of downtown Los Angeles, as previously reported¹⁵. Constituent components of the particles were analyzed as described³⁴. The respective particles were suspended in an aqueous solution, with the hydrophilic components becoming part of the solution, while the solid non-soluble UFP cores are left in suspension. The entire mixture was administered intranasally, as indicated below

T cell co-cultures with lung macrophages and DC. Nave CD4⁺DO11.10⁺ T cells were isolated from spleens of CD4⁺DO11.10⁺RAG2^{-/- Il}4ra^{R576}Foxp3^{EGFP} mice by fluorescein-activated cell sorting (FACS). AM and IM were isolated by FACS and were aliquoted at 2×10⁴ cells in 48 well plates, then either sham treated or treated overnight with UFP at 10 μg/ml. The UFP treatment did not induce increased apoptosis as compared to sham treatment, as assessed by Annexin V staining (data not shown). The APC were washed twice with PBS to remove residual UFP, and the T cells were then added at 4×10⁵ cells/well in a final volume of 0.5 ml 10% fetal calf serum/RPMI culture medium. Cultures were treated with the OVA₃₂₃₋₃₃₉ peptide at as indicated. Anti-murine Notch Ab were added at 10 μg/ml each, as indicated.

Allergic sensitization and challenge. Mice were sensitized to OVA by intraperitoneal (i.p.) injection of 100 μg OVA in 100 μl PBS, then boosted two weeks later with a second i.p. injection of OVA in PBS. Control mice were sham sensitized and boosted with PBS alone. Starting on day 29, both OVA and sham-sensitized mice were challenged with aerosolized OVA at 1%, for 30 minutes daily for 3 days. Two hours before each OVA aerosol exposure, subgroups of mice were given intransally (i.n.) either PBS or UFP at 10 μg/100μl PBS/instillation. For anti-Notch4 antibody blocking, 150 μg Armenian hamster anti-mouse Notch4 IgG mAb (clone HMN4-14; Bio X Cell)³⁵, or control Armenian hamster IgG polyclonal antibodies (Ab) (Bio X cell), were suspended in 100 μl PBS buffer and administered daily for three consecutive days during OVA aerosol challenge. Mice were euthanized on day 32 post sensitization and analyzed. For dust mite-induced allergic airway inflammation, mice received 5 μg of lyophilized D. Pteronyssinus extract (Greer) in 100 μl PBS intranasally for 3 days at the start of the protocol then challenged with the same dose of D. Pteronyssinus extract on days 15-17 with or without UFP. Mice were euthanized on day 18 and analyzed for measures of airway inflammation. Bronchoalveolar lavage (BAL) fluid and lung tissues was obtained and analyzed for cellular components and T cell cytokine expression following previously published methods 15,30.

For AM, IM and DC cell transfer studies, cells were isolated from either Il4ra^{R576} or Il4ra^{R576}Lyz2^{Cre}Jag1^{Δ/Δ} donor mice by FACS. The cells were cultured overnight and either sham treated, or loaded with OVA₃₂₃₋₃₃₉ peptide at 5 nM peptide concentration either alone or together with UFP at 10 μg/ml. The cells were transferred intratracheally to OVA-sensitized Il4ra^{R576}Lyz2^{Cre}Jag1^{Δ/Δ} recipient mice at 10⁵ cells/mouse repeated twice over two days. The mice were euthanized on the third day and analyzed for the different parameters of allergic airway inflammation.

Lung histopathology staining. Paraffin-embedded lung sections were stained with hemtoxylin and eosin (H&E) as described³⁶. The lung pathology was scored by blinded operators. Inflammation was scored separately for cellular infiltration around blood vessels and airways: 0, no infiltrates; 1, few inflammatory cells; 2, a ring of inflammatory cells 1 cell layer deep; 3, a ring of inflammatory cells 2-4 cells deep; 4, a ring of inflammatory cells of >4 cells deep³⁷. A composite score was determined by the adding the inflammatory scores for both vessels and airways. The number and distribution of goblet cells was assessed by Periodic Acid Schiff (PAS) staining of mucin granules. Individual airways (bronchi/bronchioles) were scored for goblet cell hyperplasia according to the following scale: 0, no PAS-positive cells; 1, <5% PAS-positive cells; 2, 5 to 10% PAS-positive cells; 3, 10 to 25% PAS-positive cells; and 4, >25% PAS-positive cells³⁸.

Statistical analysis. Student's two-tailed t-test, one and two way ANOVA and repeat measures two way ANOVA with Bonferroni post-test analysis of groups were used to compare test groups, as indicated. A p value <0.05 was considered statistically significant.

Study approval. All animal studies were reviewed and approved by the Boston Children's Hospital office of Animal Care Resources.

Other Methods. Information real time PCR analysis, flow cytometry (including Fluoresbrite® yellow green (YG) microspheres and nanobeads), intracellular staining reagents, Ab and methods, IgE ELISA and measurement of airway hyper-responsiveness are provided in the Methods section in this article's Online Repository at, e.g., which can be found on the world wide web at www.jacionline.org.

Results

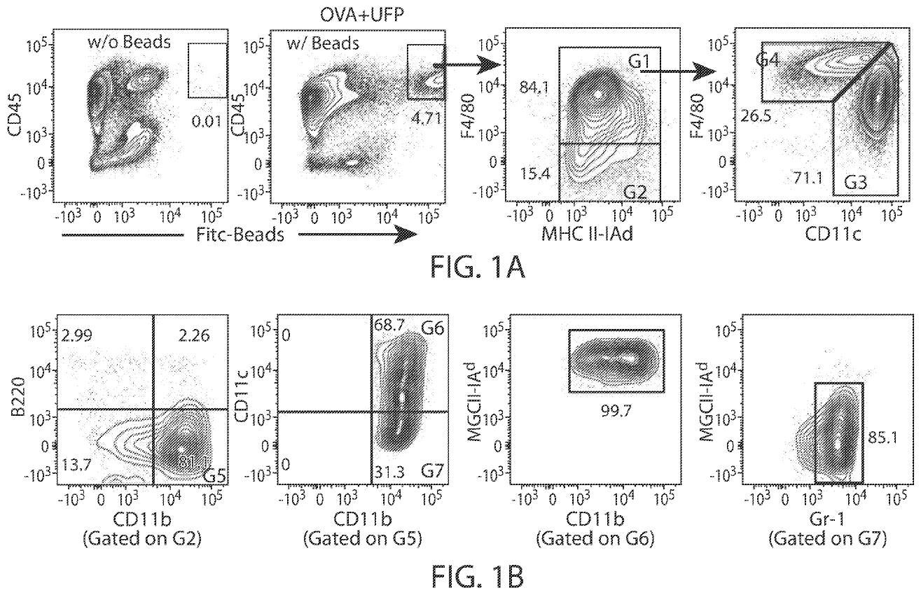

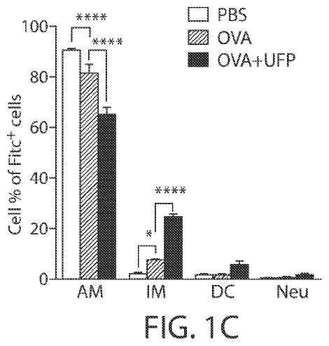

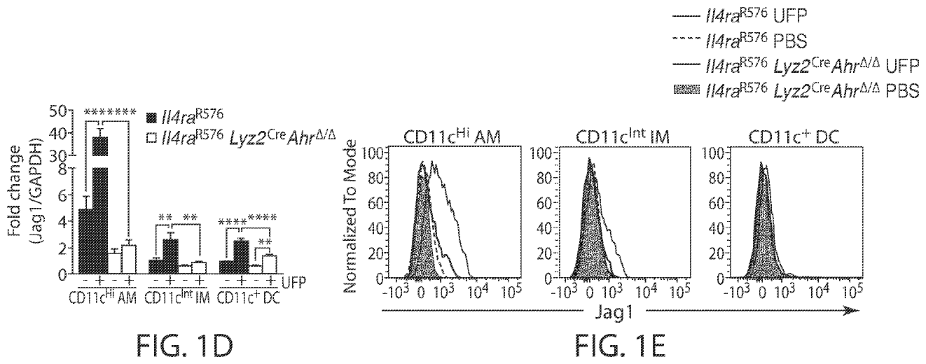

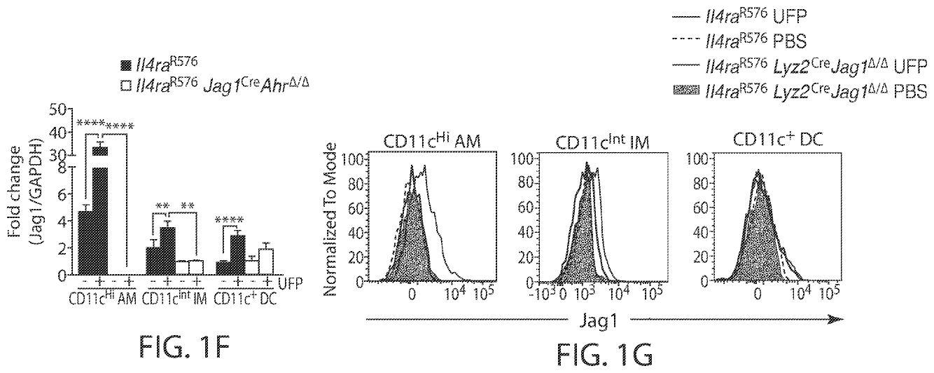

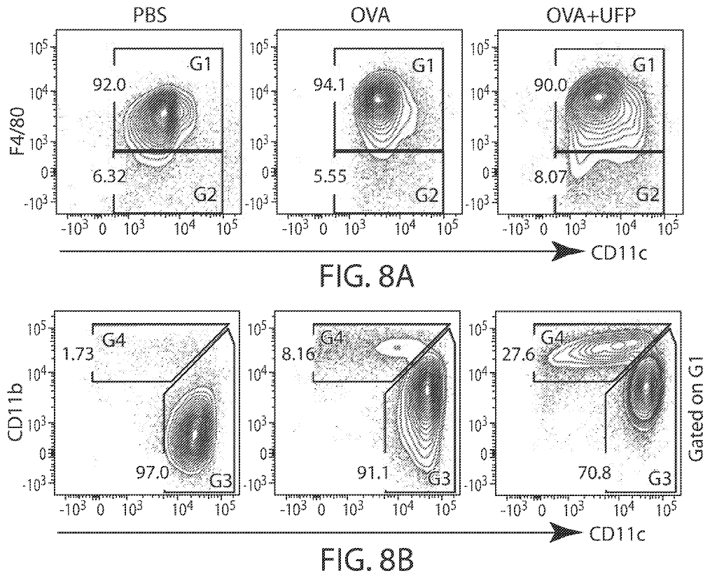

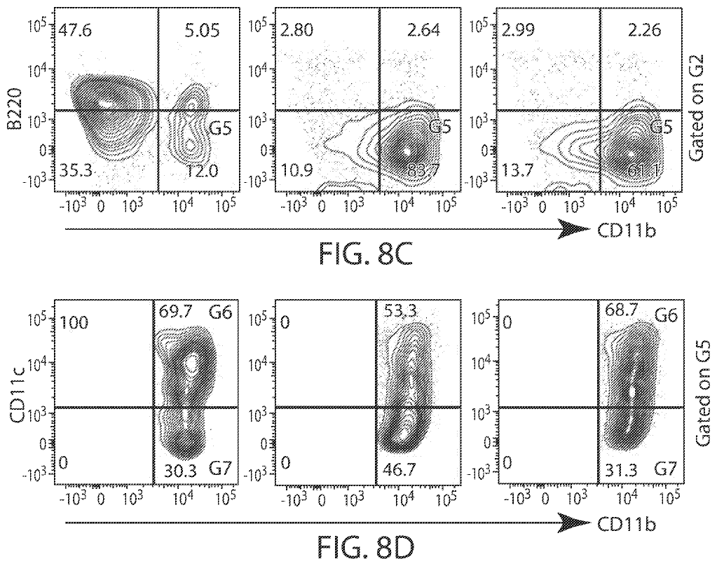

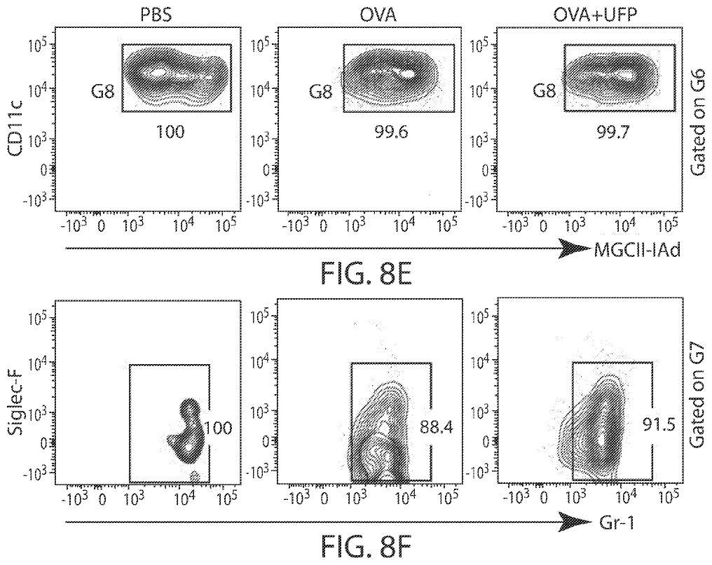

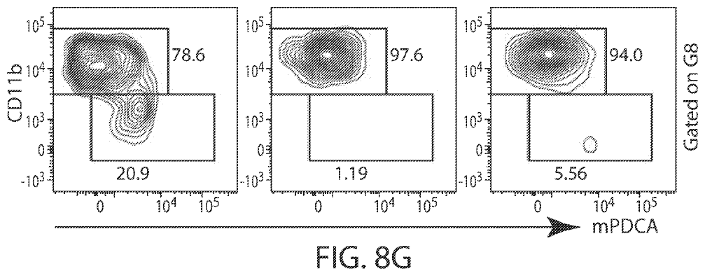

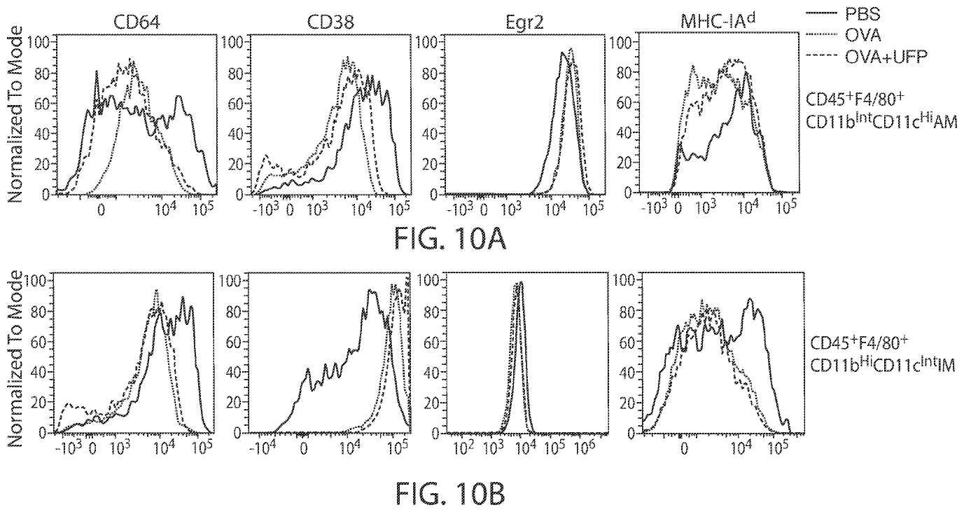

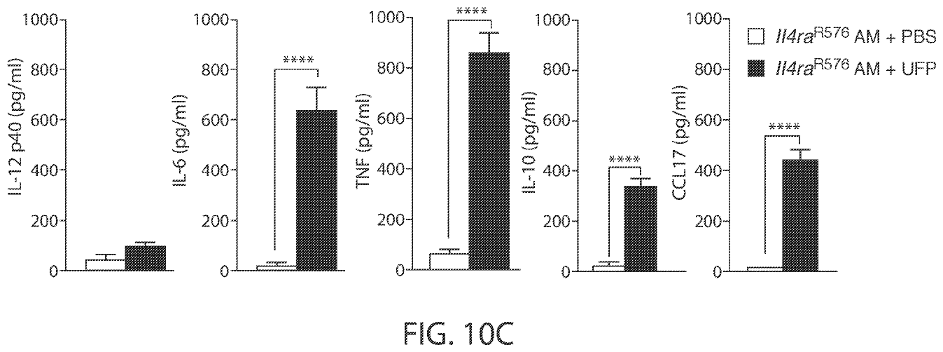

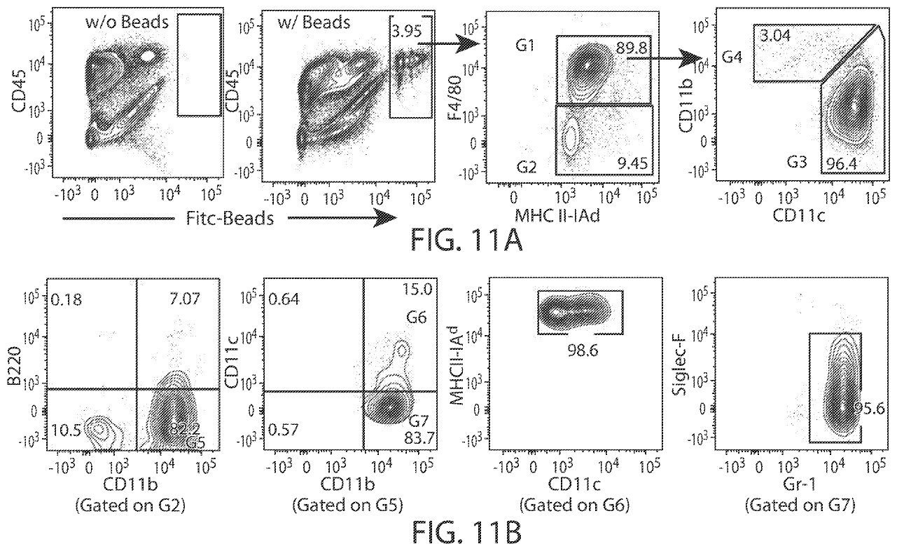

Lung Macrophages are the major cellular target of UFP in the lungs under basal and inflammatory conditions. To further elucidate the role of the Jag1-AhR-Notch pathway in the promotion of airway inflammation by UFP, it was first sought to establish detailed immunophenotypic characterization of the APC targeted by PM in the study model presented herein, both under basal and especially allergic inflammatory conditions. The studies on UFP were primarily focused on, which are particularly toxic by virtue of their deep penetrance, large surface area to size ratios, higher content per mass of PAH and greater capacity to induce oxidative stress 39, 40. An OVA-induced allergic airway inflammation model using the Il4raR576 mice was used. These mice carry an IL-4 receptor alpha chain (IL-4Rα-R576) variant that mediates exaggerated allergic airway inflammation to allergen, alone or in combination with UFP, by virtue of mediating IL-4R-dependent mixed Th2/Th17 cell inflammation 15, 30. Mice were sensitized by intra-peritoneal injection of OVA and then challenged by repeated inhalation of PBS (sham challenge) or 1% aerosolized OVA. Subgroups of mice were treated intra-nasally with UFP in combination with Fluoresbrite® YG nanobeads (0.05 μm effective diameter) or microspheres (1 μm effective diameter) 2 hr before the OVA aerosol challenge 41. Nanobead fluorescence positive cell populations were analyzed by flow cytometry (FIGS. 1A and 1B). In the absence of inflammation (PBS-sensitized, OVA-treated group), ≥95% nanobead-uptaking cells were CD45+F4/80+CD64+MHCII+CD11bIntCD11cHi alveolar macrophage whereas CD45+F4/80+CD64+MHCII+CD11bHiCD11cInt interstitial macrophage represented 2-3% of total microsphere positive cells (FIGS. 8A-8G, 9A-9D, and 10A-10C) 42. In the context of allergic airway inflammation induced by OVA and especially by OVA+UFP, about 85% of nanobeads-uptaking cells were macrophages, distributed at a 70:30 ratio between AM (gate G3) and IM (gate G4) (FIG. 1A-1C). The particle-uptaking AM population was CD38IntEgr2Hi (M2-like), while the IM population was CD38HiEgr2Int (M1-like) (FIGS. 10A and 10B) 43. In agreement with their M2-like phenotype, in vitro treatment of cell-sorted AM with UFP sharply upregulated their production of the cytokines IL-10, CCL17, IL-6 and TNF-α but induced little change in their baseline production of IL-12 (FIG. 10C) 44, 45. Of the remaining beads (15%), about ⅔rd were picked up by CD11chiCD11bintCD8α+CD103+B220PDCA1 classical (c)DCs 46, and the rest by Gr1+SiglecFlow neutrophils (FIGS. 1B, 1C, and 8A-8G). These results indicated that alveolar and interstitial macrophage were the major cell subset responsible for the clearance of inhaled UFP in the context of allergic airway inflammation. Similar cellular localization results were obtained when Fluoresbrite® YG microsphere beads were used instead of nanobeads, consistent with previously published data showing FP and UFP sharing the same AhR-Notch-Jag1 mechanism of action in promoting allergic airway inflammation (FIG. 11A-11B).

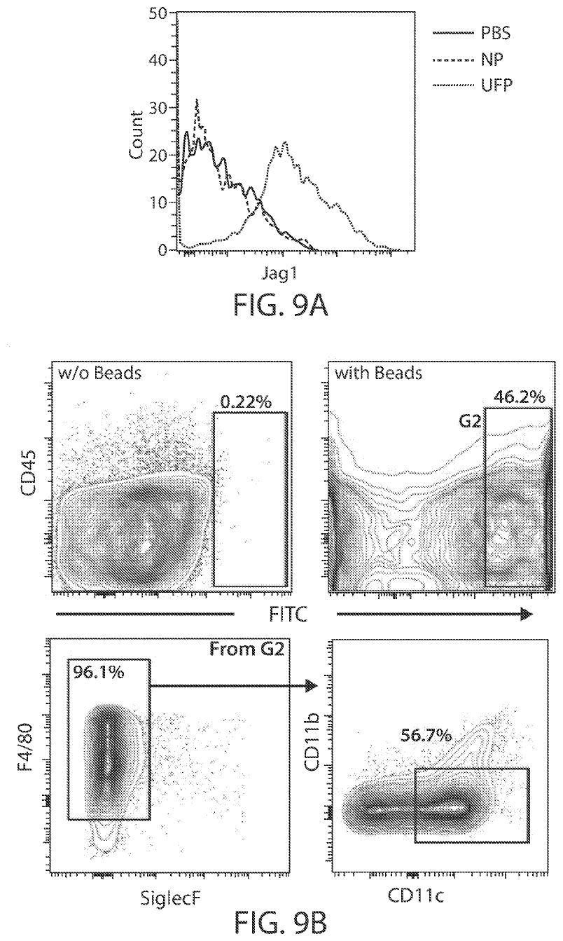

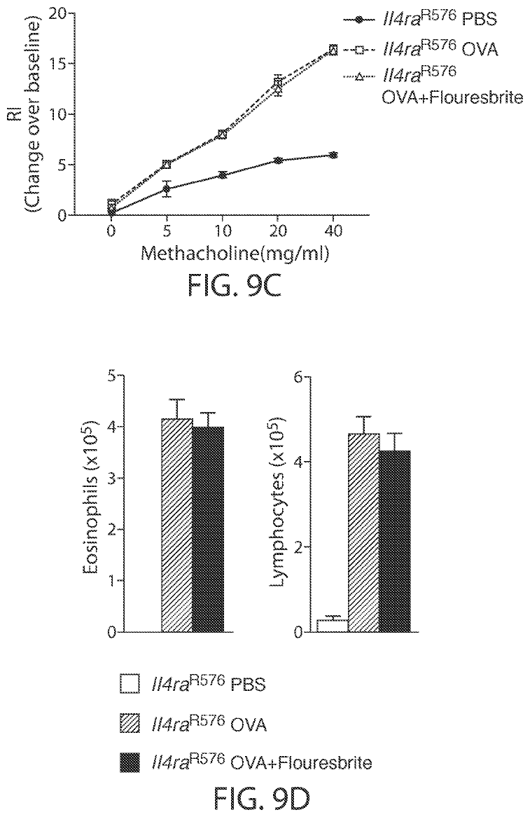

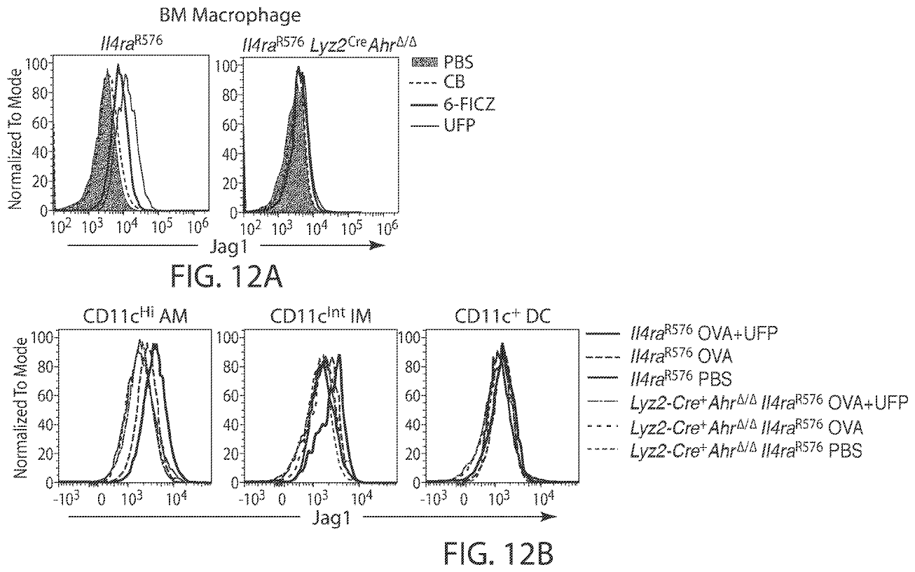

UFP differentially induce Jag1 expression in lung AM. UFP induces Jag1 expression in an AhR-dependent manner in bone marrow-derived dendritic cells 15 and in macrophages (FIG. 12A). The expression of Jag1 transcripts was examined in different APC populations isolated from the lungs of Il4raR576 mice and either sham treated or treated in vitro with UFP. Jag1 expression was highest at baseline in AM as compared to IM and DC (FIG. 1D). In vitro treatment with UFP super-induced Jag1 transcript expression in AM, whereas the same treatment was associated with modest increases in IM and DC (FIG. 1D). In contrast, treatment with Fluoresbrite® YG nanobeads failed to induce Jag1 expression on AM (FIG. 9A). It also failed to affect airway inflammation induced by OVA despite the Fluoresbrite® YG nanobeads localizing to AM when administered intranasally (FIG. 9B-9D). Deletion of a foxed Ahr allele by means of a Cre recombinase driven by the lysozyme 2 gene promoter (Lyz2Cre), which is active in myeloid-lineage cells, greatly reduced baseline expression of Jag1 transcripts in AM of Il4raR576Lyz2CreAhrΔ/Δ mice and abolished its super-induction by UFP. While a similar trend was also noted in the other cell types, there was partial sparing of Jag1 expression in DC. Flow cytometric analysis confirmed the heightened expression of Jag1 in AM as compared to the other cell types and its downregulation upon Ahr deletion (FIG. 1E). UFP-induced Jag1 expression in bone marrow-derived macrophages was similarly affected by Lyz2Cre-driven Ahr deletion (FIG. 12A-12B). Furthermore, sensitization of mice with OVA followed by challenge with OVA and UFP resulted in the preferential induction of Jag1 on AM as compared to IM and DC, and this induction was reversed upon by Lyz2Cre-driven Ahr deletion (FIG. 12B).

These results were further ascertained by the deletion of a foxed Jag1 gene in myeloid lineages using Lyz2Cre (Il4raR576Lyz2CreJag1Δ/Δ) (FIGS. 1F and 1G). Jag1 transcript expression and Jag1 surface staining were completely abrogated in AM both at baseline and following UFP treatment. Reduced Jag1 transcript expression and Jag1 protein staining persisted in IM, while their levels were unaffected in DC. These findings confirmed AM as the principal APC cell type expressing Jag1 both at baseline and following UFP treatment and that this expression proceeds by an Ahr-dependent mechanism. They also showed that Lyz2Cre preferentially targets Jag1 in macrophages, particularly AM, while largely sparing it in DC, consistent with previous lineage tracing analysis on the activity of Lyz2Cre in macrophages versus dendritic cells 47.

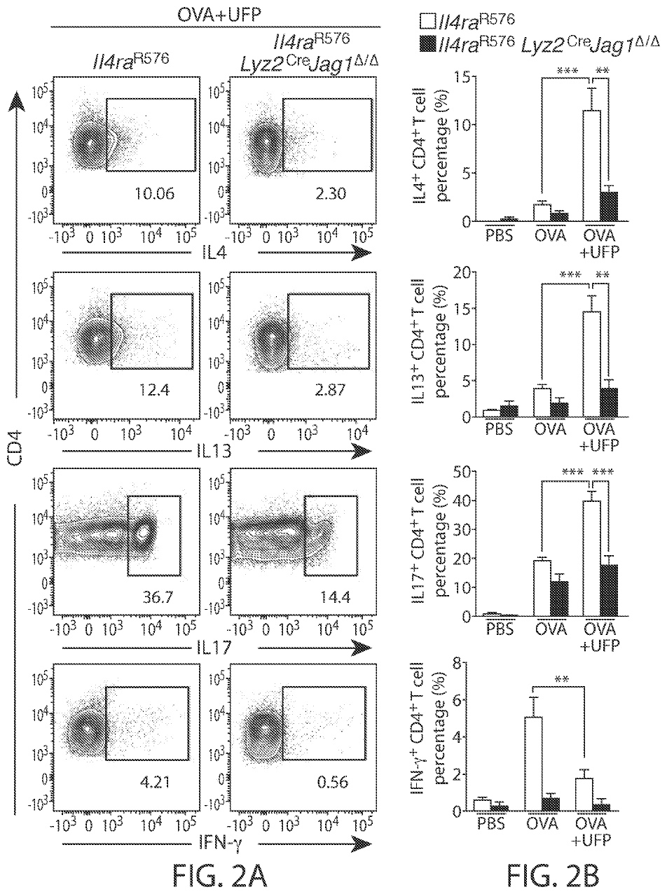

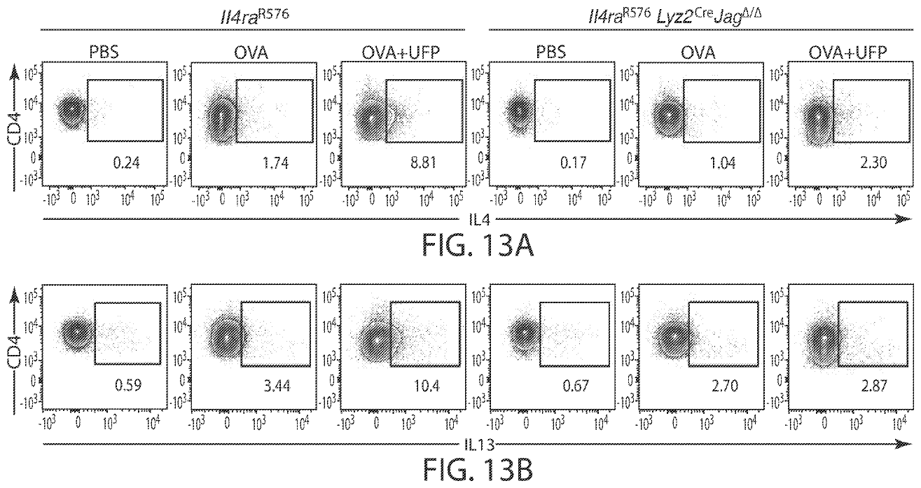

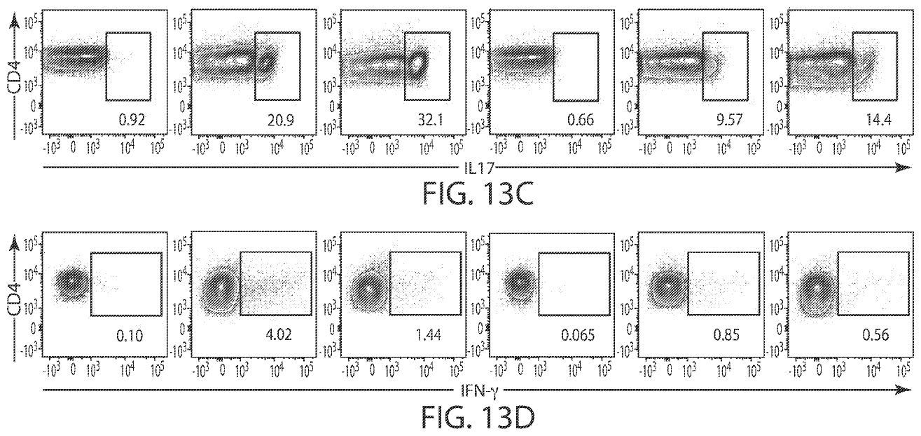

UFP-treated AM promote Th cell differentiation in a Jag1-dependent mechanism. To examine if induction of Jag1 expression in AM by UFP augments allergen-induced Th cell differentiation, an in vitro Th cell differentiation system involving naïve Il4raR576DO11.10+CD4+ T cells, derived from DO11.10+Rag2−/− mice was employed. Naïve DO11.10+CD4+ T cells were incubated with FACS-purified AM isolated from Il4R576 or Il4raR576Lyz2CreJag1Δ/Δ mice. The AM were either sham pulsed with PBS or pulsed with the OVA peptide OVA323-339, alone or together with UFP. At the end of the incubation period, Th cell cytokine expression was analyzed in gated CD4+Foxp3− (non-regulatory) T cells. Co-culture with OVA323-339 peptide-pulsed IL4RR576 AM resulted in increased production by DO11.10+CD4+Foxp3− T cells of IL-17, IL-13 and IL-4, and to much lesser extent IFN-γ, as revealed by flow cytometric staining (FIGS. 2A, 2B, and 13A-13D). Expression of the first three cytokines was markedly upregulated by the addition of UFP (10 μg/ml), whereas that of IFN-γ was down-regulated, consistent with exaggerated Th2/Th17 skewing 30. In contrast, the induction of IL-17, IL-13 and IL-4 expression in DO11.10+CD4+ T cells by OVA323-339 was moderately inhibited, and their super-induction by UFP completely abolished, when Il4raR576Lyz2CreJag1Δ/Δ AM were used as APC. IFN-γ expression in those co-cultures was also profoundly impaired (FIGS. 2A and 2B).

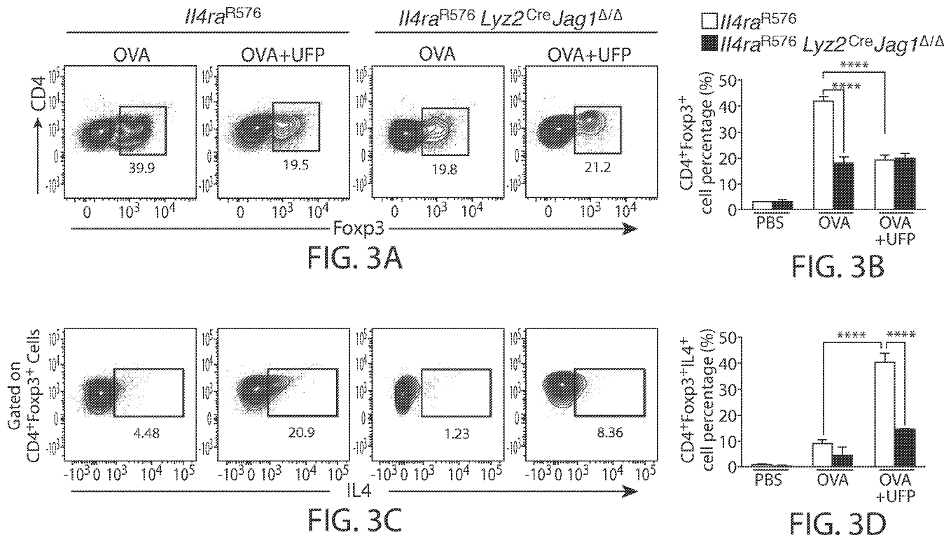

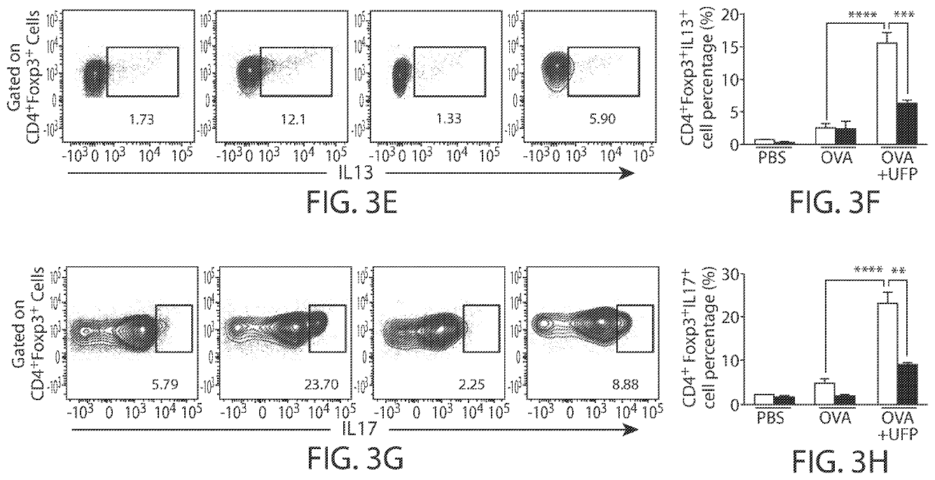

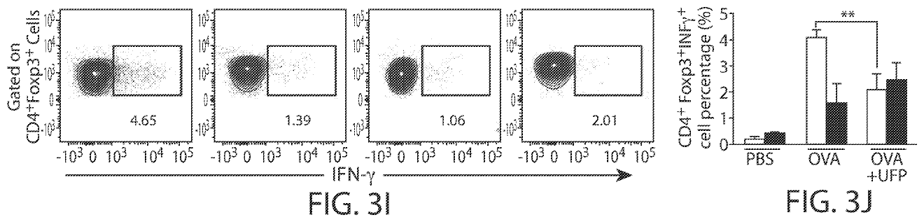

The DO11.10 cell in vitro Th cell differentiation system was also employed to examine the impact of UFP treatment on the capacity of AM to support the differentiation of naïve allergen-specific T cells into induced Treg cells. In the absence of UFP, OVA323-339-loaded AM drove the differentiation of up to 40% of naïve Il4raR576DO11.10+CD4+ T cells into Foxp3+ induced T regulatory (iTreg) cells. Treatment with UFP partially inhibited iTreg cell differentiation independent of Jag1 expression (FIGS. 3A and 3B). Critically however, UFP treatment of OVA323-339 peptide-presenting AM skewed the formed iTreg cells into secreting Th2/17 cell cytokines, including IL-4, IL-13 and IL-17, but not the Th1 cytokine IFN-γ. This skewing was largely reversed by deletion of Jag1 in AM (FIGS. 3C and 3D). These results indicated that UFP adversely affected allergen-specific iTreg cell differentiation, in part by destabilizing newly formed iTreg cells towards Th2/17 cell differentiation in a Jag1-dependent manner.

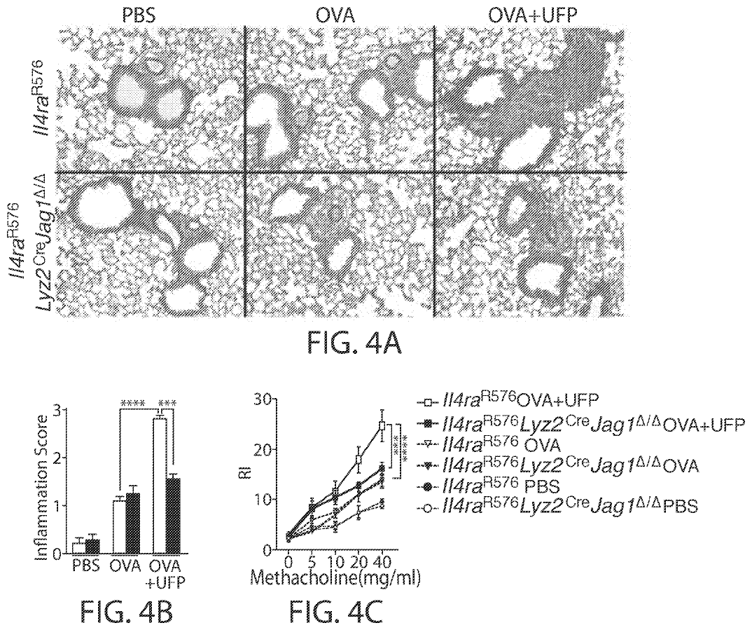

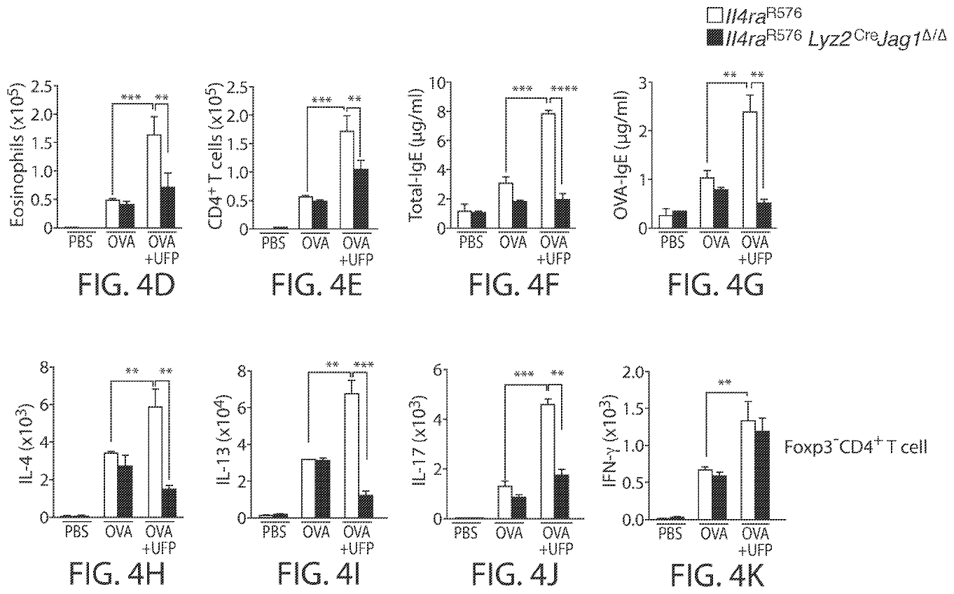

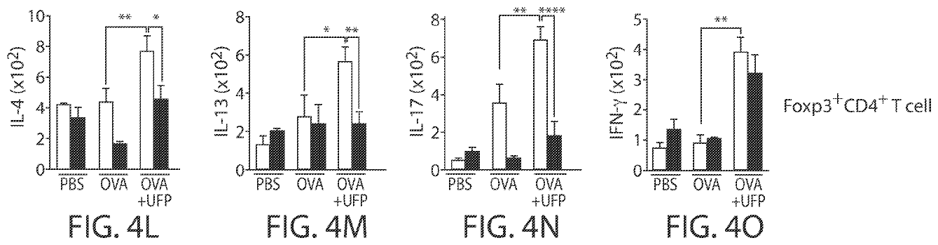

Jag1 deletion in myeloid lineages abolishes the augmentation of allergic airway inflammation by UFP. To determine the role of Jag1 expression on AM in supporting UFP upregulation of allergic airway inflammation in vivo, Lyz2Cre was first employed to delete component genes of the Ahr-Jag1 genetic circuit in myeloid lineage cells. Accordingly, Il4raR576Lyz2CreJag1Δ/Δ mice and Il4raR576 mice were sensitized by intra-peritoneal injection of OVA and then challenged by inhalation of 1% aerosolized OVA. Control mice were sham sensitized with PBS and challenged with aerosolized OVA. Subgroups of mice were treated intra-nasally with UFP (10 μg/instillation) or PBS 2 hr before the OVA aerosol challenge 41. Sensitization and challenge of Il4raR576 mice with OVA resulted in a robust airway inflammatory response, characterized by airway inflammation and hyper-responsiveness, eosinophilia and T cell infiltration in the BAL fluid, elevated total and OVA-specific serum IgE responses, and augmented Th2 and Th17 cell responses (FIG. 4A-4K). All these parameters were markedly augmented by UFP exposure during the OVA challenge phase. OVA sensitization and challenge of Il4raR576Lyz2CreJag1Δ/Δ mice also resulted in a robust allergic airway inflammatory response that was similar to that noted in OVA sensitized and challenged Il4raR576 mice. However, the augmentation by UFP of all the aforementioned parameters of allergic airway inflammation was completely abrogated in Il4raR576Lyz2CreJag1Δ/Δ mice, indicating a requisite requirement for Jag1 expression in myeloid lineages for UFP to exert its pro-inflammatory effects in the airways (FIG. 4A-4I).

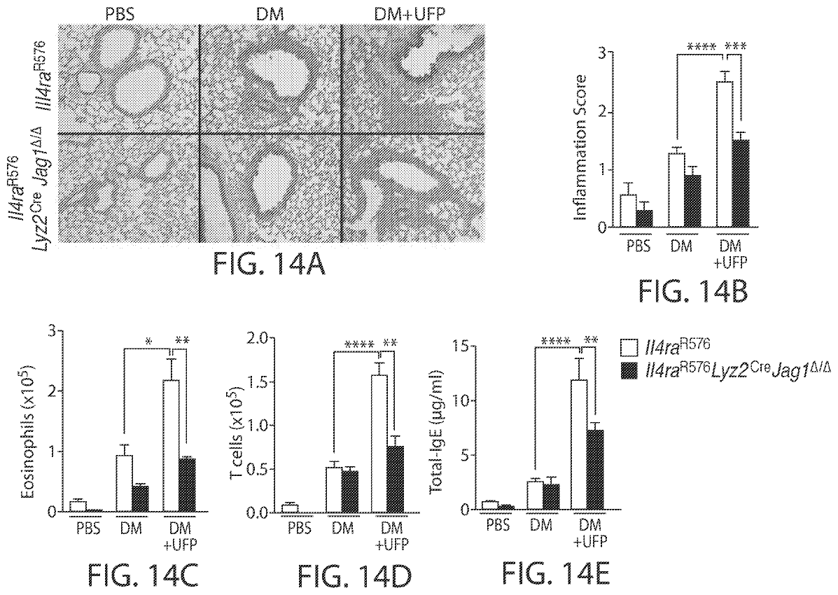

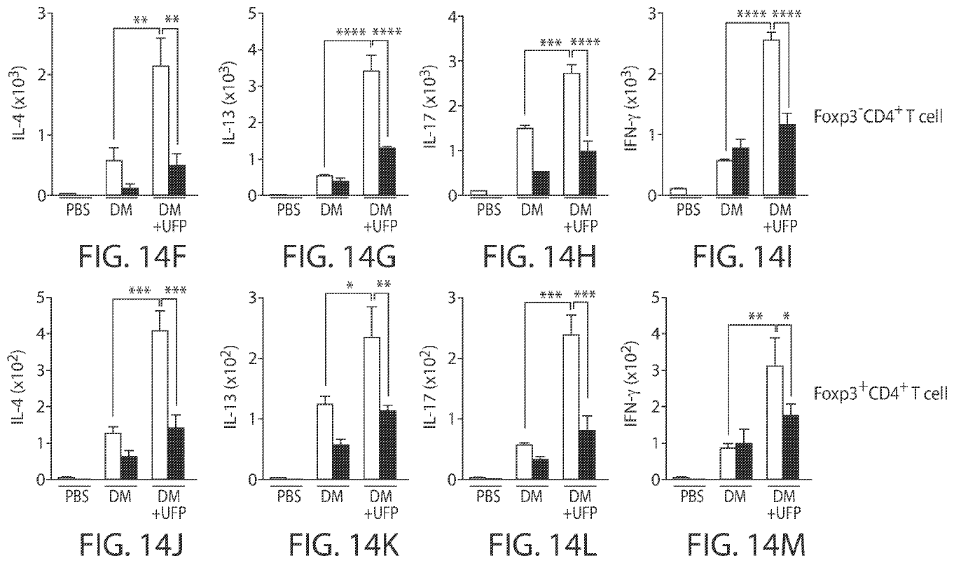

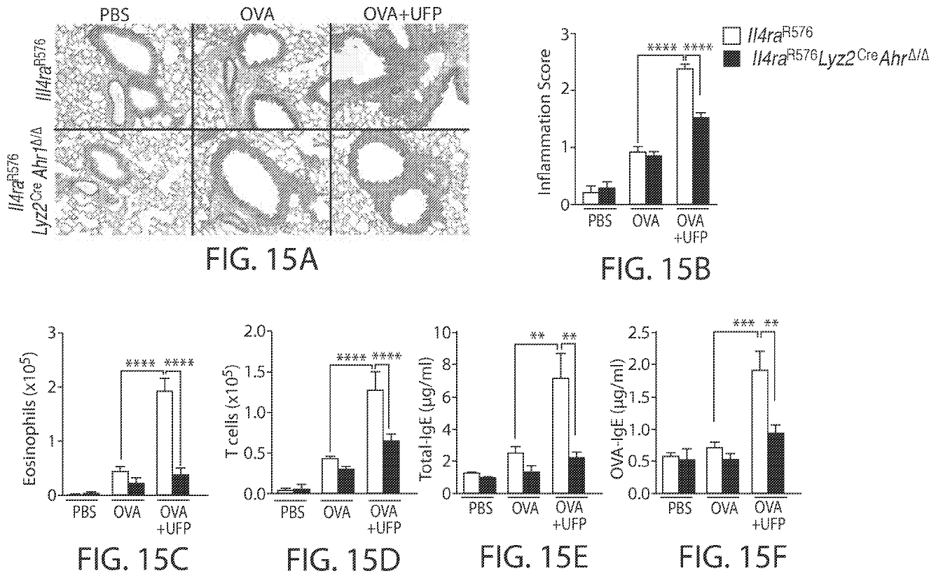

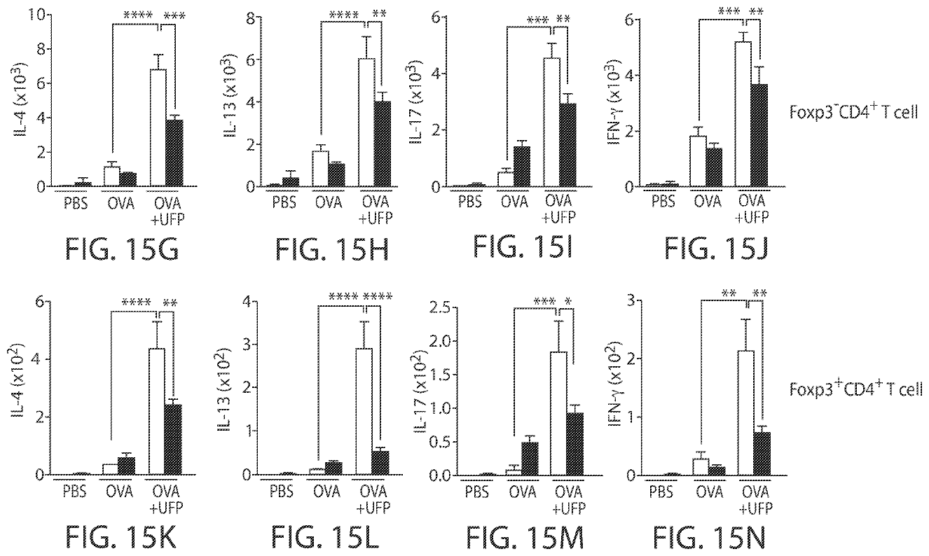

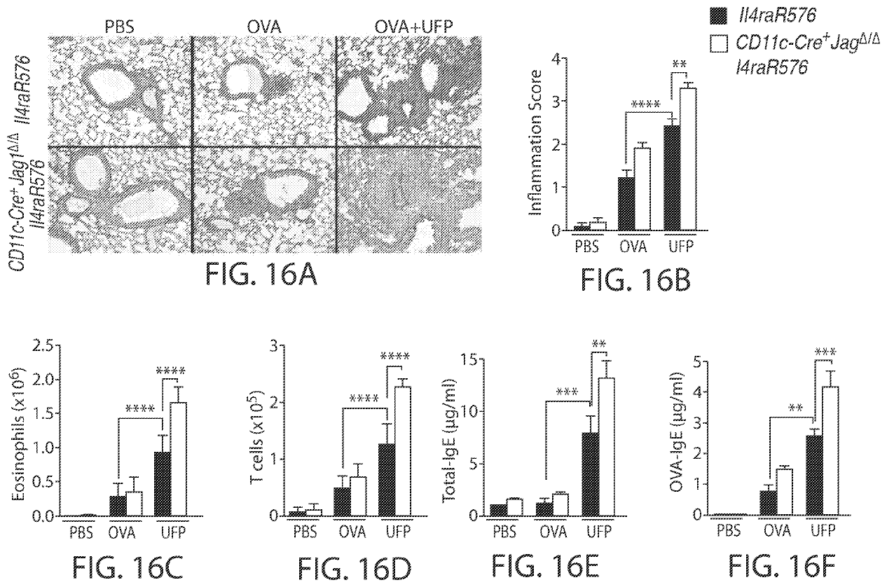

The role of Jag1 expression in myeloid lineage cells to mediate the augmentation by UFP of allergic airway inflammation induced upon the intranasal treatment of Il4raR576 mice was also examined with extracts of house mites (D. pteronyssinus), a common and potent human allergen. Results were concordant with those observed with OVA sensitization and challenge. Whereas UFP augmented the airway inflammation induced by treatment of Il4raR576 mice with a low dose of D. pteronyssinus (5 μg), this effect was abrogated in Il4raR576Lyz2CreJag1Δ/Δ mice (FIG. 14A-14M). Myeloid lineage-specific deletion of Ahr in Il4raR576 mice (Il4raR576Lyz2CreAhrΔ/Δ) also abrogated the capacity of UFP to augment the various parameters of allergic airway inflammation induced by OVA, consistent with the requirement for AhR signaling for the induction of Jag1 expression by UFP (FIG. 15A-15N). In contrast, deletion of Jag1 in all CD11c+ APC lineages using a CD11cCre did not inhibit the promotion of airway inflammation by UFP and in fact worsened it, indicating a unique and specific requirement for Jag1 induction by UFP in AM for their acquisition of a pro-inflammatory function (FIGS. 16A-16F).

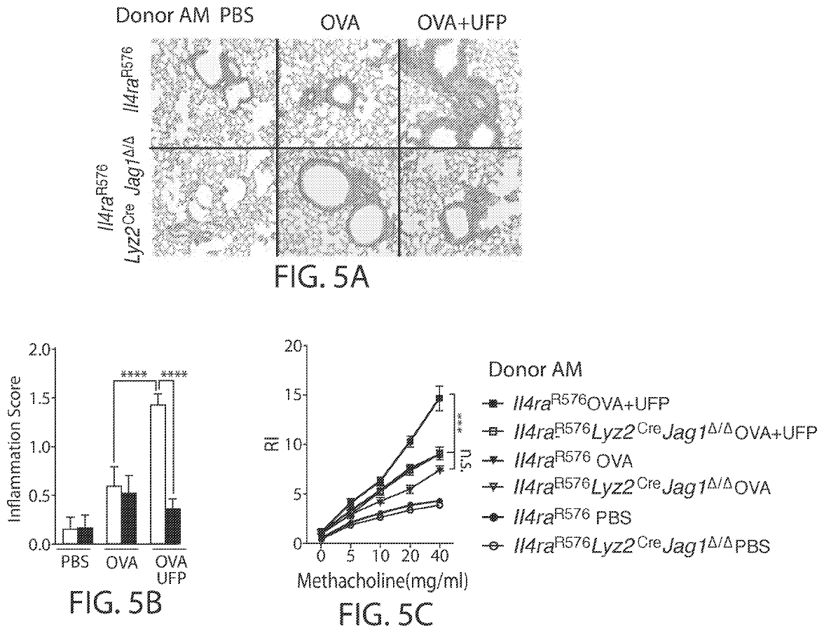

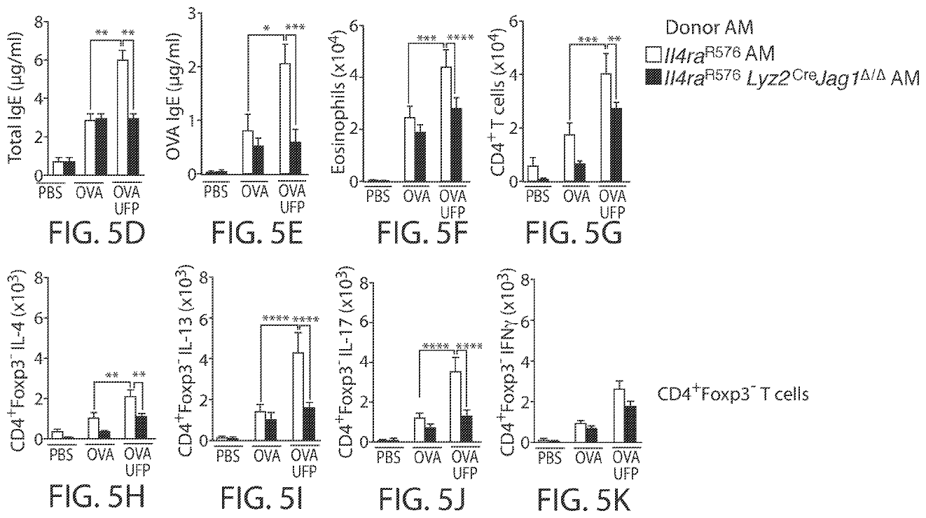

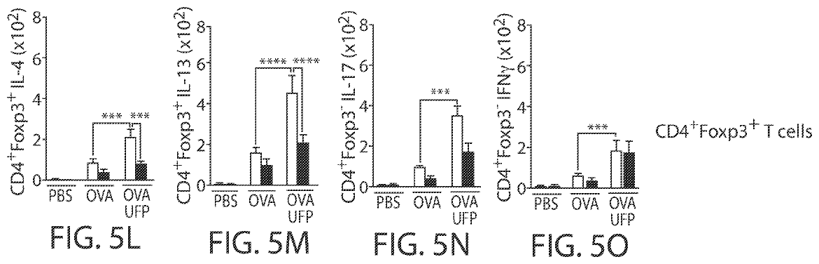

Jag1 expression in AM is sufficient to mediate UFP upregulation of allergic airway inflammation. To specifically establish the role of Jag1 expression in AM in the exacerbation of allergen-induced airway inflammation by UFP, the capacity of AM to rescue the UFP effect when transferred into Il4raR576Lyz2CreJag1Δ/Δ mice was examined. Accordingly, AM were isolated from either Il4raR576 or Il4raR576Lyz2CreJag1Δ/Δ mice, either sham treated or loaded with OVA323-339 peptide in the absence or presence of UFP. The cells were transferred into the airways of Il4raR576Lyz2CreJag1Δ/Δ mice that were sensitized with OVA, which were then examined for induction of allergic airway inflammation. Results revealed that transfer of Jag1-sufficient OVA323-339 peptide-pulsed and UFP-treated Il4raR576 AM into OVA-sensitized Il4raR576Lyz2CreJag1Δ/Δ mice recapitulated all the stigmata of exacerbated allergic airway inflammation induced by UFP, including augmented tissue inflammation, increased airway hyper-responsiveness, serum total and OVA-specific IgE, and BAL fluid CD4+ T cell infiltration and eosinophilia (FIG. 5A-5G). It also augmented Th cell cytokine production and Treg cell destabilization into Th cell like phenotypes (FIG. 5H-5O). In contrast, transfer of similarly treated Jag1-deficient Il4raR576Lyz2CreJag1Δ/Δ AM failed to do so, indicating that Jag1 expression of AM is sufficient to restore the capacity of UFP to augment allergen-induced airway inflammation.

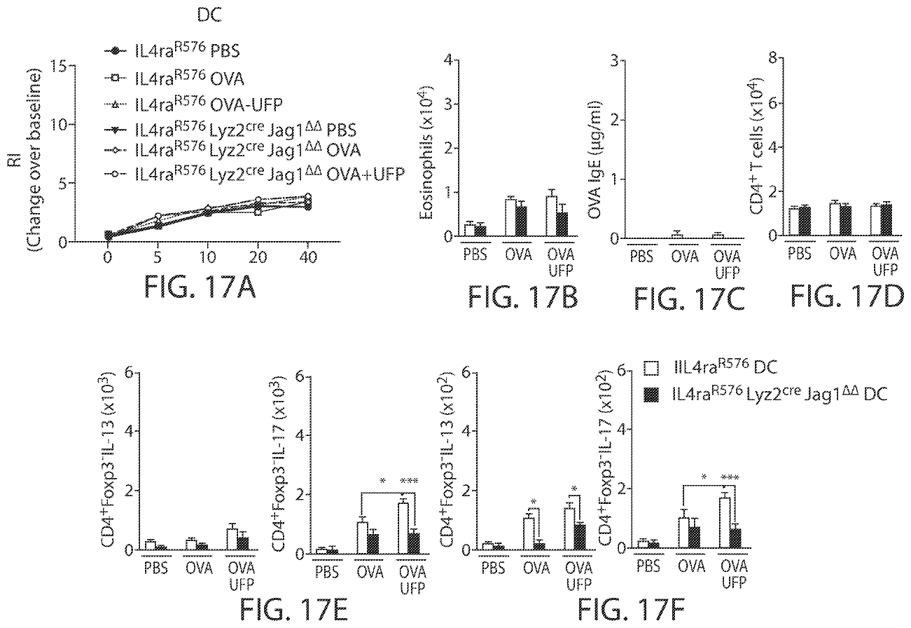

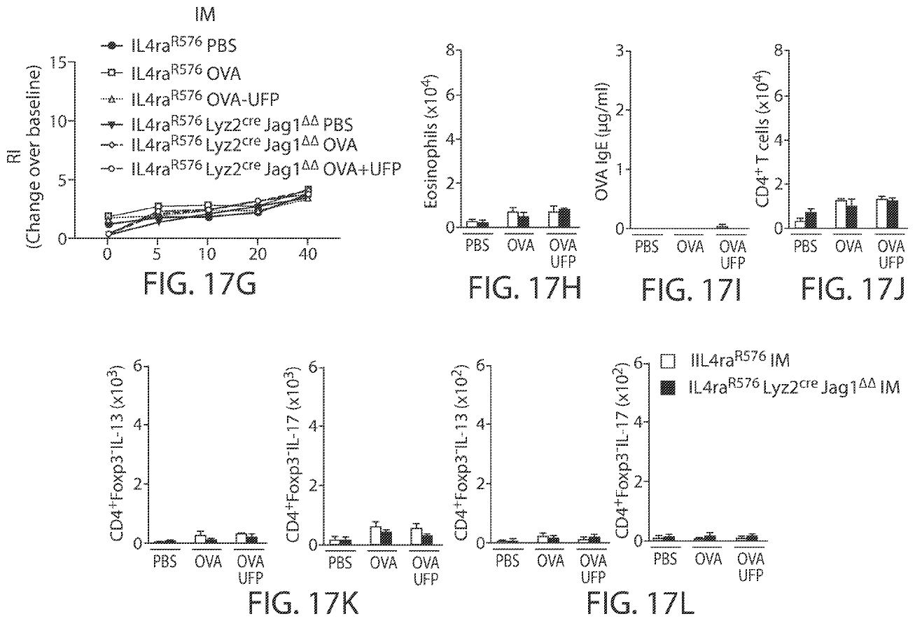

The capacity of IM and DC isolated from the lungs of Il4raR576 or Il4raR576Lyz2CreJag1Δ/Δ mice and sham treated or loaded with OVA323-339 or OVA323-339+UFP to promote airway inflammation was also examined when transferred intra-tracheally into OVA-sensitized Il4raR576Lyz2CreJag1Δ/Δ mice. However, unlike the case of Jag1-sufficient AM, transferred IM failed to promote allergic airway inflammation irrespective of their treatment modality (FIG. 17A-17F). The transferred DC also failed to induced airway hyper-responsiveness. OVA323-339 loaded DC induced suboptimal tissue infiltration with eosinophils and Th cells as compared to AM, which was not augmented by UFP treatment. These results are consistent with the unique role of Jag1-sufficient AM in promoting airway inflammation and in its super-induction by UFP (FIG. 17G-17L).

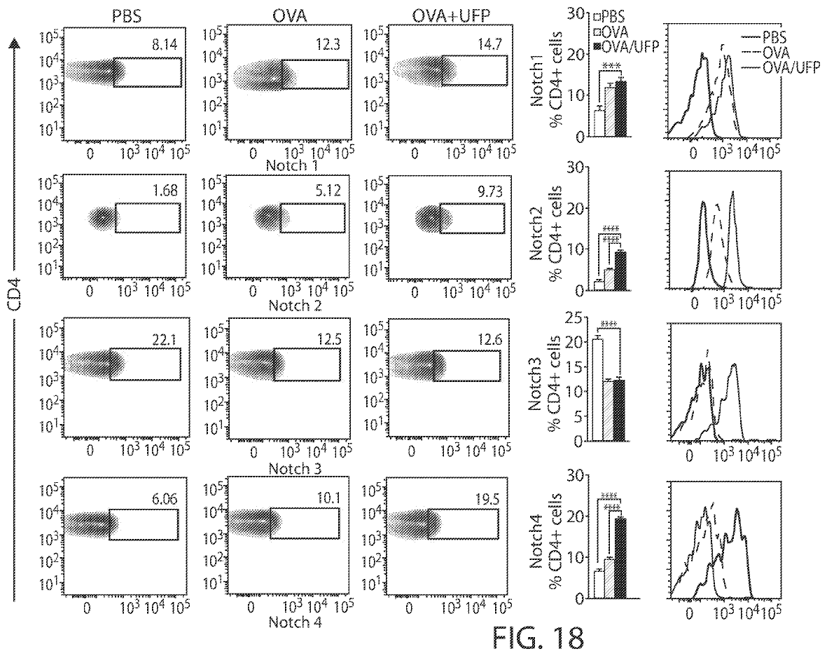

Promotion by UFP of allergen-induced Th cell differentiation involves Jag1-Notch4 interaction. The role of the respective Notch receptors in mediating the pro-inflammatory effects of Jag1 expressed by AM upon UFP treatment was next determined. Accordingly, the ex-vivo expression of the different Notch receptors was first analyzed in FACS-purified CD4+EGFP T conventional cells and CD4+EGFP+ Treg cells, isolated from Il4raR576Foxp3EGFP mice. The cells were isolated from the spleens of unmanipulated mice and from the lungs of mice subjected to allergic airway inflammation without or with UFP co-treatment. Splenic CD4+ T cells primarily expressed Notch1 and Notch2 (data not shown), consistent with previous results 48. In contrast, Notch4 expression was upregulated in CD4+ T cells isolated from the lungs of mice sensitized with OVA and challenged with OVA+UFP, to become the highest among the four Notch receptors (FIG. 18). Notch1 and Notch2 expression was also upregulated but to a lesser extent, while that of Notch3 decreased.

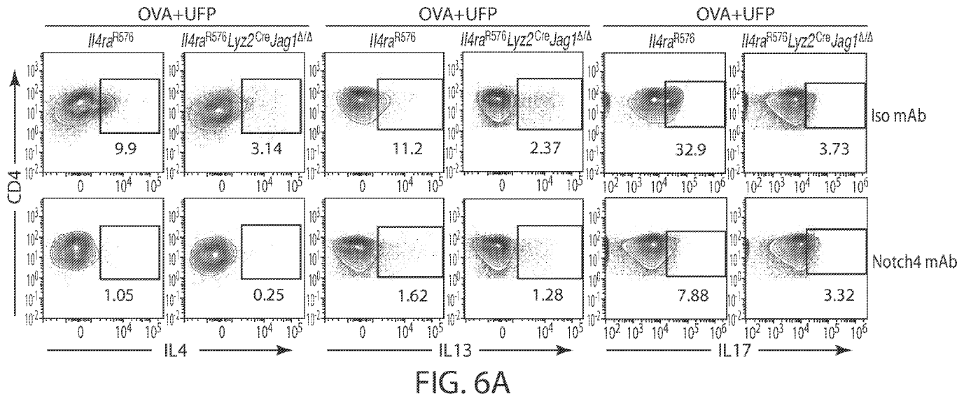

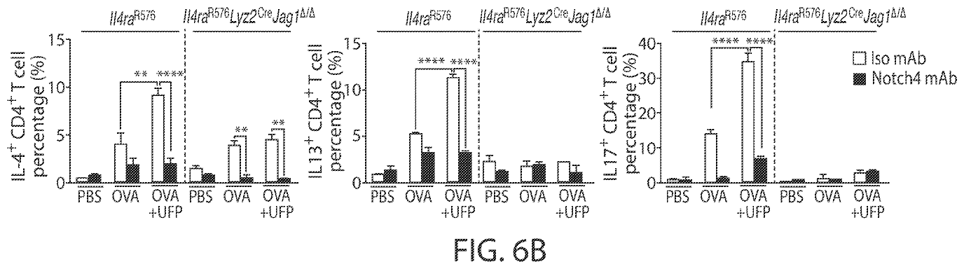

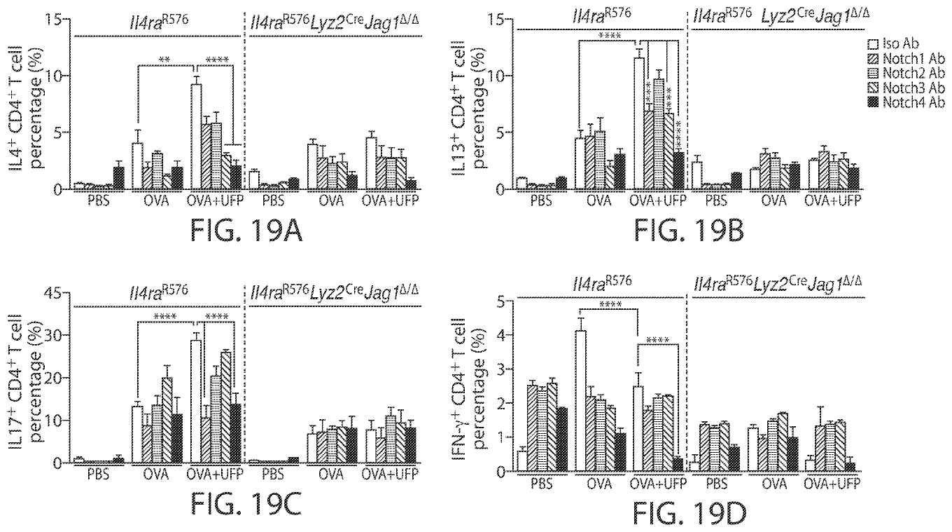

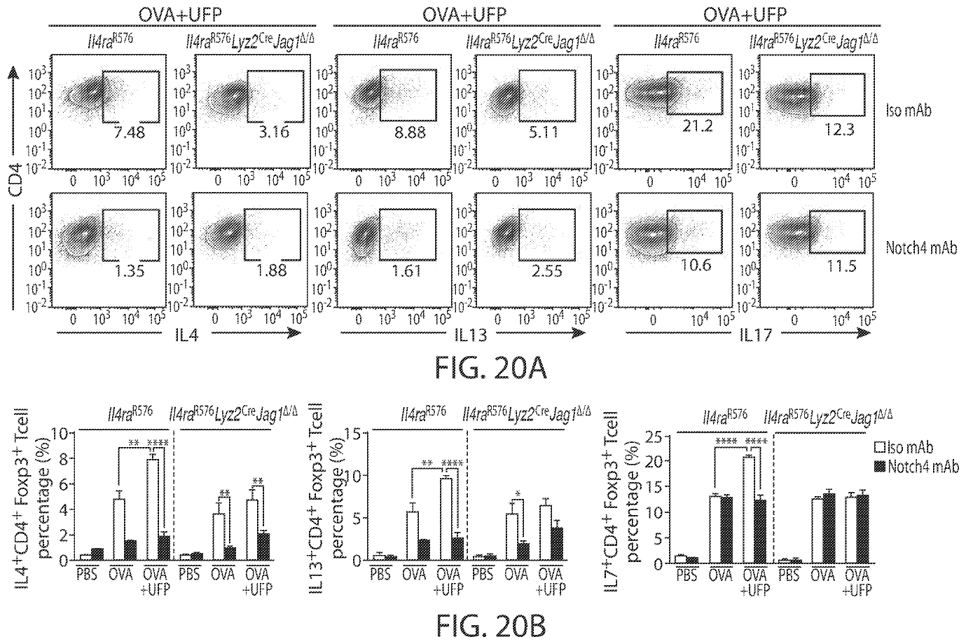

Informed by the above results, the in vitro co-culture system described in FIGS. 2A and 2B was next employed to determine the capacity of neutralizing Notch1, 2 and 4 Ab to reverse the augmentation by UFP treatment of OVA323-339 peptide-presenting AM of Th cell cytokine production by responding DO11.10 T cells. FACS-purified Jag1-sufficient (Il4raR576) or -deficient (Il4raR576Lyz2CreJag1Δ/Δ) AM were either sham treated or treated with OVA323-339 peptide, either alone or together with UFP. They were co-cultured with nave Il4raR576DO11.10+CD4+ T cells in the presence of either isotype control mAbs or neutralizing mAbs specific for individual Notch receptors, and the T cells were examined for Th cell cytokine expression. As expected, UFP treatment of OVA323-339 peptide-pulsed Il4raR576 AM upregulated the production by DO11.10+CD4+Foxp3− T cells of IL-17, IL-13 and IL-4, and to much lesser extent IFN-γ, whereas this effect was abolished when Il4raR576Lyz2CreJag1Δ/Δ AM were used as APC. Critically, upregulation of Th cell cytokine expression by UFP, including IL-4, IL-13 and IL-17, was uniformly inhibited by co-treatment with a highly specific neutralizing anti-Notch4 mAb (FIGS. 6A and 6B, and 19A-19D) 49. Anti-Notch4 mAb also inhibited the residual IFN-γ production induced by OVA, alone or with UFP. In contrast, treatment with neutralizing mAbs specific for other Notch receptors gave partial and/or selective inhibitory results (FIG. 19A-19D). Anti-Notch4 mAb also suppressed the production by DO11.10+CD4+Foxp3+iTreg cells of Th cell cytokines when cultured with OVA323-339 peptide-presenting AM that were treated with UFP (FIG. 20A-20B). Of note, treatment with the anti-Notch4 occasionally suppressed residual Th cell cytokine production (e.g. IL-4 production) beyond what could be accounted for by Jag1 activation, indicating an additional contribution by other Notch ligands acting via Notch4 in supporting those Th cell responses.

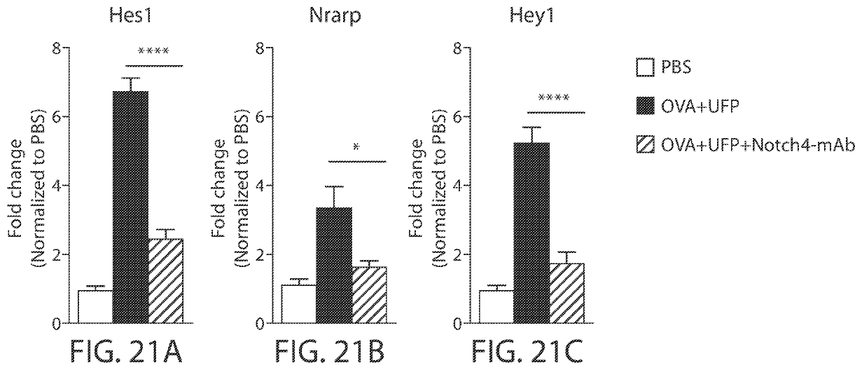

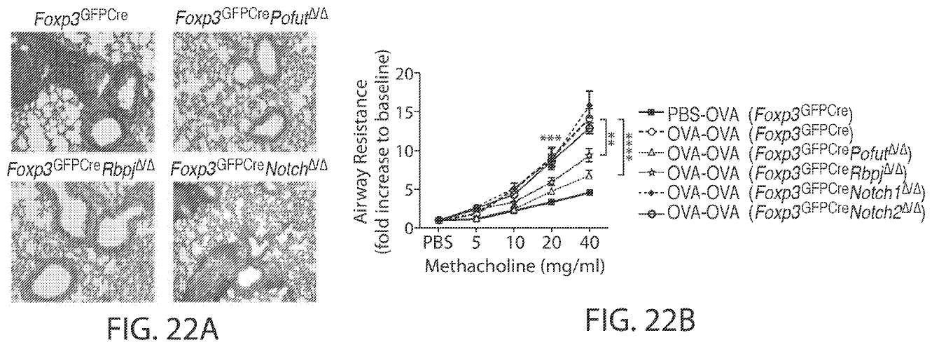

The specificity and efficacy of the anti-Notch4 mAb in blocking Notch signaling in allergen-specific T cells was further ascertained in in vitro co-cultures of OVA323-339 and UFP-treated AM with Il4raR576CD4+DO11.10+Rag2/T cells, in which treatment with anti-Notch4 mAb blocked the transcriptional upregulation of the Notch target genes Hes1, Hey1 and Nrarp (FIG. 21A-21C). Overall, these findings presented herein indicated Notch4 as a key Notch receptor through which Jag1 mediates the inflammatory responses to UFP by promoting Th cell differentiation.

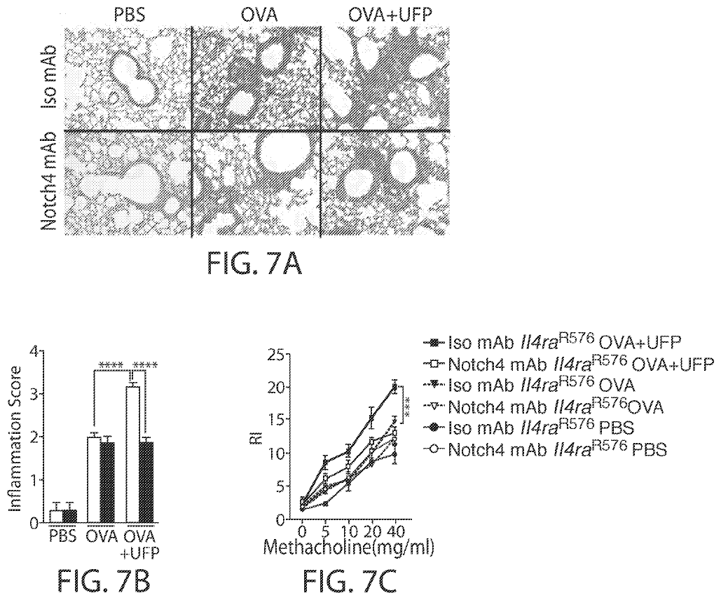

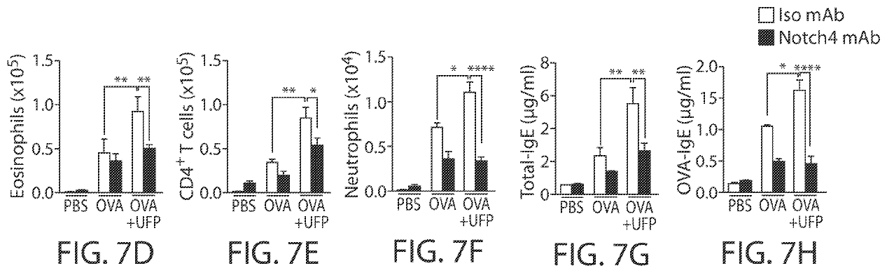

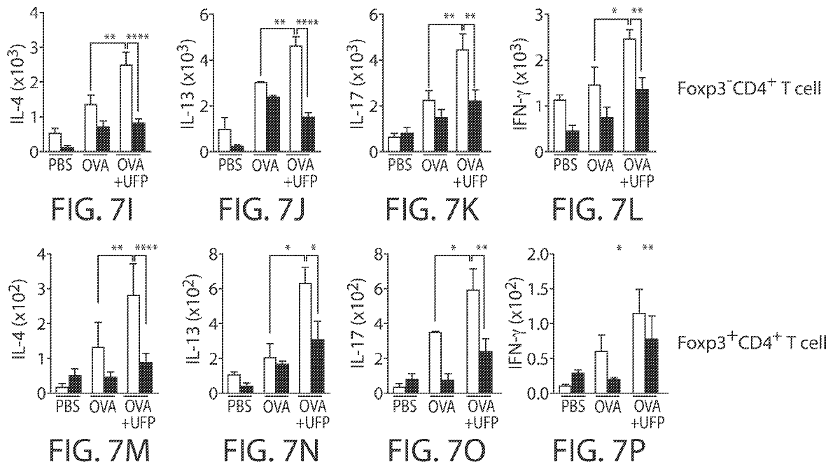

Notch4 inhibition suppresses the exacerbation of allergic airway inflammation by UFP. Given the efficacy of the neutralizing anti-Notch4 mAb in reversing the augmented in vitro differentiation of allergen-specific Th cells induced by UFP treatment of allergen peptide-presenting AM, the impact of inhibiting Notch4 on the exacerbation of the allergic airway inflammatory response induced by UFP was examined. Accordingly, Il4raR576 mice sensitized and challenged with OVA alone or together with UFP, were treated with either an anti-Notch4 or an isotype control mAb during the challenge phase then analyzed for the various parameters of the airway allergic inflammatory response, treatment with the anti-Notch4 mAb had little or no effect on OVA-induced allergic airway inflammation in terms of tissue inflammation, airway hyper-responsiveness, BAL fluid eosinophilia, serum total and OVA-specific IgE response, and airway Th2 and Th17 cell responses. In contrast, it completely inhibited the potentiation of the aforementioned parameters induced by UFP, thus indicating Notch4 in mediating the potentiating effects of UFP on allergic airway inflammation (FIG. 7A-7G).

Discussion

Previous studies have demonstrated that traffic-related PM, including UFP and FP, promotes allergic airway inflammation by inducing Jag1 expression on APCs in an AhR-dependent manner, which in turn activates Notch signaling to augment Th cytokine expression by allergen-specific T cells¹⁵. However, these studies do not teach that Notch4 as a potential therapeutic target for attenuating airway inflammation. Data presented herein has identified AM as the key target of PM by virtue of their avid uptake of nano and micro particles and their super-induction of Jag1 expression upon PM uptake as compared to other lung APCs. Additionally, presented herein is the identification of Notch4 on T cells as a key mediator of the Jag1-dependent upregulation by UFP-treated AM of allergen-specific Th cell differentiation and iTreg cell destabilization. Notch4 inhibition by means of a neutralizing anti-Notch4 mAb completely abrogated the upregulation by UFP of allergic airway inflammation. These studies thus established cellular elements, including AM and allergen-specific CD4⁺ Th and Treg cells, and molecular effectors, including Jag1 and Notch4, involved in mediating the pro-inflammatory effects of the PM-activated AhR-Jag1-Notch circuit in allergic airway inflammation.

AM have been indicated in the homeostatic maintenance of tolerance in the airways by virtue of their down regulation of the antigen presenting capacity of DC⁵⁰, as well as their promotion of iTreg cell differentiation²³. AM are also less effective in presenting antigens as compared to DC, a defect that could be overcome by the provision of an accessory signal such as co-stimulation of T cells with CD28 or IL-2^{51,52}. Upregulation of Jag1 expression in AM by PM-mediated activation of AhR may enable efficient antigen presentation with Jag1-Notch acting as a co-receptor pair that amplifies Th cell cytokine production⁵³. Results presented herein clearly demonstrate a necessary and sufficient role for Jag1-sufficient AM to rescue the augmentation by UFP of allergic airway inflammation in mice lacking Jag1 in their myeloid lineages. Under inflammatory conditions, increased uptake of nanoparticles by IM was noted, possibly reflecting in part the increased abundance of the latter cells in inflamed lung tissues and/or their heightened avidity for these particles. Nevertheless, reconstitution of Il4ra^{R576}Lyz2^{Cre}Jag1^{Δ/Δ} mice with either IM or DC failed to rescue the inflammatory responses to UFP, indicative of the requisite role of Jag1-sufficient AM in this process.

While deletion of Jag1 on AM reversed UFP-induced augmentation of allergic airway inflammation, it also attenuated a few parameters of allergen-induced airway inflammation in the absence of UFP, such as the mobilization of CD4⁺ T cell in lung tissues (FIGS. 4A-4O and 5A-5O). These findings argue that in the absence of UFP treatment residual Jag1 expression on AM may contributes to allergen-induced airway inflammation, and that UFP acts to greatly amplify this process.

Surprisingly, findings presented herein identified of Notch4 as a key Notch receptor through which UFP-mediated their effects in upregulating allergic airway inflammation. Notch4 inhibition provided effective and uniform suppression of UFP and AM-dependent in vitro differentiation of allergen-specific T cells into different Th cell subsets. In contrast, inhibition of other Notch receptors, including, including Notch1 and Notch2, provided selective and/or partial inhibition of Th cell cytokine expression. Notch4 inhibition also suppressed the exacerbation by UFP of allergic airway inflammation in mice. The NOTCH4 locus has previously been associated with severe asthma⁵⁴, indicating that this pathway may modulate disease severity, especially in as it relates to environmental exposures such as to UFP.

Jag1 expressed on AM may preferentially interact with Notch4 as compared to other Notch receptors. Alternatively or in parallel, Notch4 can act to differentially amplify the production of Th cell cytokines, or can instruct their specific production, as compared to other Notch receptors by means of Notch canonical and non-canonical signaling mechanisms^{53,55}. Notch4 signaling also destabilized differentiating of iTreg cells, leading to their production of Th cell cytokines. Such an iTreg cell phenotype is associated with decreased suppressive function and lineage instability, potentially leading to the terminal differentiation of Treg cells into Th cell lineages^{30,56}. Distinct, dedicated functions of different Notch receptors in Th and Treg cell populations in allergic airway inflammation may offer opportunities for targeted therapeutic interventions. Finally, and in addition to targeting T cells, a neutralizing Notch4 mAb can act to modulate additional cellular elements, such as the vascular endothelium, involved in mobilizing the airway inflammatory response^{57,58}.

### REFERENCES

1. Brandt E B, Myers J M, Ryan P H, Hershey G K. Air pollution and allergic diseases. Curr Opin Pediatr 2015; 27:724-35.
2. Bowatte G, Lodge C, Lowe A J, Erbas B, Perret J, Abramson M J, et al. The influence of childhood traffic-related air pollution exposure on asthma, allergy and sensitization: a systematic review and a meta-analysis of birth cohort studies. Allergy 2015; 70:245-56.
3. Gehring U, Wijga A H, Hoek G, Bellander T, Berdel D, Bruske I, et al. Exposure to air pollution and development of asthma and rhinoconjunctivitis throughout childhood and adolescence: a population-based birth cohort study. Lancet Respir Med 2015; 3:933-42.
4. Adar S D, Filigrana P A, Clements N, Peel J L. Ambient Coarse Particulate Matter and Human Health: A Systematic Review and Meta-Analysis. Curr Environ Health Rep 2014; 1:258-74.
5. Khreis H, Kelly C, Tate J, Parslow R, Lucas K, Nieuwenhuijsen M. Exposure to traffic-related air pollution and risk of development of childhood asthma: A systematic review and meta-analysis. Environ Int 2017; 100:1-31.
6. Brunst K J, Ryan P H, Brokamp C, Bernstein D, Reponen T, Lockey J, et al. Timing and Duration of Traffic-related Air Pollution Exposure and the Risk for Childhood Wheeze and Asthma. Am J Respir Crit Care Med 2015; 192:421-7.
7. Dockery D W, Pope C A, 3rd. Acute respiratory effects of particulate air pollution. Annu Rev Public Health 1994; 15:107-32.
8. Brunekreef B, Holgate S T. Air pollution and health. Lancet 2002; 360:1233-42.
9. Saxon A, Diaz-Sanchez D. Air pollution and allergy: you are what you breathe. Nat Immunol 2005; 6:223-6.
10. Nel A. Atmosphere. Air pollution-related illness: effects of particles. Science 2005; 308:804-6.
11. Diaz-Sanchez D, Dotson A R, Takenaka H, Saxon A. Diesel exhaust particles induce local IgE production in vivo and alter the pattern of IgE messenger RNA isoforms. J Clin Invest 1994; 94:1417-25.
12. Diaz-Sanchez D, Tsien A, Fleming J, Saxon A. Combined diesel exhaust particulate and ragweed allergen challenge markedly enhances human in vivo nasal ragweed-specific IgE and skews cytokine production to a T helper cell 2-type pattern. J Immunol 1997; 158:2406-13.
13. Li N, Harkema J R, Lewandowski R P, Wang M, Bramble L A, Gookin G R, et al. Ambient ultrafine particles provide a strong adjuvant effect in the secondary immune response: implication for traffic-related asthma flares. Am J Physiol Lung Cell Mol Physiol 2010; 299:L374-83.
14. Brandt E B, Kovacic M B, Lee G B, Gibson A M, Acciani T H, Le Cras T D, et al. Diesel exhaust particle induction of IL-17A contributes to severe asthma. J Allergy Clin Immunol 2013; 132:1194-204 e2.
15. Xia M, Viera-Hutchins L, Garcia-Lloret M, Noval Rivas M, Wise P, McGhee S A, et al. Vehicular exhaust particles promote allergic airway inflammation through an aryl hydrocarbon receptor-notch signaling cascade. J Allergy Clin Immunol 2015.
16. Oberdorster G. Pulmonary effects of inhaled ultrafine particles. Int Arch Occup Environ Health 2001; 74:1-8.
17. Brandt E B, Biagini Myers J M, Acciani T H, Ryan P H, Sivaprasad U, Ruff B, et al. Exposure to allergen and diesel exhaust particles potentiates secondary allergen-specific memory responses, promoting asthma susceptibility. J Allergy Clin Immunol 2015; 136:295-303 e7.
18. Hiraiwa K, van Eeden S F. Contribution of lung macrophages to the inflammatory responses induced by exposure to air pollutants. Mediators Inflamm 2013; 2013:619523.
19. Hardy C L, Lemasurier J S, Mohamud R, Yao J, Xiang S D, Rolland J M, et al. Differential uptake of nanoparticles and microparticles by pulmonary APC subsets induces discrete immunological imprints. J Immunol 2013; 191:5278-90.
20. Blank F, Stumbles P A, Seydoux E, Holt P G, Fink A, Rothen-Rutishauser B, et al. Size-dependent uptake of particles by pulmonary antigen-presenting cell populations and trafficking to regional lymph nodes. Am J Respir Cell Mol Biol 2013; 49:67-77.
21. Garbi N, Lambrecht B N. Location, function, and ontogeny of pulmonary macrophages during the steady state. Pflugers Arch 2017; 469:561-72.
22. Kopf M, Schneider C, Nobs S P. The development and function of lung-resident macrophages and dendritic cells. Nat Immunol 2015; 16:36-44.
23. Coleman M M, Ruane D, Moran B, Dunne P J, Keane J, Mills K H. Alveolar macrophages contribute to respiratory tolerance by inducing FoxP3 expression in naive T cells. Am J Respir Cell Mol Biol 2013; 48:773-80.
24. Soroosh P, Doherty T A, Duan W, Mehta A K, Choi H, Adams Y F, et al. Lung-resident tissue macrophages generate Foxp3+ regulatory T cells and promote airway tolerance. J Exp Med 2013; 210:775-88.
25. Duan W, So T, Croft M. Antagonism of airway tolerance by endotoxin/lipopolysaccharide through promoting OX40L and suppressing antigen-specific Foxp3+T regulatory cells. J Immunol 2008; 181:8650-9.
26. Moon K A, Kim S Y, Kim T B, Yun E S, Park C S, Cho Y S, et al. Allergen-induced CD11b+CD11c(int) CCR3+ macrophages in the lung promote eosinophilic airway inflammation in a mouse asthma model. Int Immunol 2007; 19:1371-81.
27. Miyata R, van Eeden S F. The innate and adaptive immune response induced by alveolar macrophages exposed to ambient particulate matter. Toxicol Appl Pharmacol 2011; 257:209-26.
28. de Haar C, Kool M, Hassing I, Bol M, Lambrecht B N, Pieters R. Lung dendritic cells are stimulated by ultrafine particles and play a key role in particle adjuvant activity. J Allergy Clin Immunol 2008; 121:1246-54.
29. Tachdjian R, Mathias C, Al Khatib S, Bryce P J, Kim H S, Blaeser F, et al. Pathogenicity of a disease-associated human IL-4 receptor allele in experimental asthma. J Exp Med 2009; 206:2191-204.
30. Massoud A H, Charbonnier L M, Lopez D, Pellegrini M, Phipatanakul W, Chatila T A. An asthma-associated IL4R variant exacerbates airway inflammation by promoting conversion of regulatory T cells to TH17-like cells. Nat Med 2016; 22:1013-22.
31. Walisser J A, Glover E, Pande K, Liss A L, Bradfield C A. Aryl hydrocarbon receptor-dependent liver development and hepatotoxicity are mediated by different cell types. Proc Natl Acad Sci USA 2005; 102:17858-63.
32. Clausen B E, Burkhardt C, Reith W, Renkawitz R, Forster I. Conditional gene targeting in macrophages and granulocytes using LysMcre mice. Transgenic Res 1999; 8:265-77.
33. Mancini S J, Mantei N, Dumortier A, Suter U, MacDonald H R, Radtke F. Jagged1-dependent Notch signaling is dispensable for hematopoietic stem cell self-renewal and differentiation. Blood 2005; 105:2340-2.
34. Shirmohammadi F, Hasheminassab S, Saffari A, Schauer J J, Delfino R J, Sioutas C. Fine and ultrafine particulate organic carbon in the Los Angeles basin: Trends in sources and composition. Sci Total Environ 2016; 541:1083-96.
35. Murata A, Yoshino M, Hikosaka M, Okuyama K, Zhou L, Sakano S, et al. An evolutionary-conserved function of mammalian notch family members as cell adhesion molecules. PLoS One 2014; 9:e108535.
36. Blaeser F, Bryce P J, Ho N, Raman V, Dedeoglu F, Donaldson D D, et al. Targeted inactivation of the IL-4 receptor alpha chain I4R motif promotes allergic airway inflammation. J Exp Med 2003; 198:1189-200.
37. Ford J G, Rennick D, Donaldson D D, Venkayya R, McArthur C, Hansell E, et al. 11-13 and IFN-gamma: interactions in lung inflammation. J Immunol 2001; 167:1769-77.
38. McMillan S J, Xanthou G, Lloyd C M. Manipulation of allergen-induced airway remodeling by treatment with anti-TGF-beta antibody: effect on the Smad signaling pathway. J Immunol 2005; 174:5774-80.
39. Li N, Wang M, Bramble L A, Schmitz D A, Schauer J J, Sioutas C, et al. The adjuvant effect of ambient particulate matter is closely reflected by the particulate oxidant potential. Environ Health Perspect 2009; 117:1116-23.
40. Li N, Georas S, Alexis N, Fritz P, Xia T, Williams M A, et al. A work group report on ultrafine particles (American Academy of Allergy, Asthma & Immunology): Why ambient ultrafine and engineered nanoparticles should receive special attention for possible adverse health outcomes in human subjects. J Allergy Clin Immunol 2016; 138:386-96.
41. Whitekus M J, Li N, Zhang M, Wang M, Horwitz M A, Nelson S K, et al. Thiol antioxidants inhibit the adjuvant effects of aerosolized diesel exhaust particles in a murine model for ovalbumin sensitization. J Immunol 2002; 168:2560-7.
42. Misharin A V, Morales-Nebreda L, Mutlu G M, Budinger G R, Perlman H. Flow cytometric analysis of macrophages and dendritic cell subsets in the mouse lung. Am J Respir Cell Mol Biol 2013; 49:503-10.
43. Jablonski K A, Amici S A, Webb L M, Ruiz-Rosado Jde D, Popovich P G, Partida-Sanchez S, et al. Novel Markers to Delineate Murine M1 and M2 Macrophages. PLoS One 2015; 10:e0145342.
44. Mantovani A, Sica A, Sozzani S, Allavena P, Vecchi A, Locati M. The chemokine system in diverse forms of macrophage activation and polarization. Trends Immunol 2004; 25:677-86.
45. Katakura T, Miyazaki M, Kobayashi M, Herndon D N, Suzuki F. CCL17 and IL-10 as effectors that enable alternatively activated macrophages to inhibit the generation of classically activated macrophages. J Immunol 2004; 172:1407-13.
46. Merad M, Sathe P, Helft J, Miller J, Mortha A. The dendritic cell lineage: ontogeny and function of dendritic cells and their subsets in the steady state and the inflamed setting. Annu Rev Immunol 2013; 31:563-604.
47. Abram C L, Roberge G L, Hu Y, Lowell C A. Comparative analysis of the efficiency and specificity of myeloid-Cre deleting strains using ROSA-EYFP reporter mice. J Immunol Methods 2014; 408:89-100.
48. Charbonnier L M, Wang S, Georgiev P, Sefik E, Chatila T A. Control of peripheral tolerance by regulatory T cell-intrinsic Notch signaling. Nat Immunol 2015; 16:1162-73.
49. Moriyama Y, Sekine C, Koyanagi A, Koyama N, Ogata H, Chiba S, et al. Delta-like 1 is essential for the maintenance of marginal zone B cells in normal mice but not in autoimmune mice. Int Immunol 2008; 20:763-73.
50. Holt P G, Oliver J, Bilyk N, McMenamin C, McMenamin P G, Kraal G, et al. Downregulation of the antigen presenting cell function(s) of pulmonary dendritic cells in vivo by resident alveolar macrophages. J Exp Med 1993; 177:397-407.
51. Blumenthal R L, Campbell D E, Hwang P, DeKruyff R H, Frankel L R, Umetsu D T. Human alveolar macrophages induce functional inactivation in antigen-specific CD4 T cells. J Allergy Clin Immunol 2001; 107:258-64.
52. Chelen C J, Fang Y, Freeman G J, Secrist H, Marshall J D, Hwang P T, et al. Human alveolar macrophages present antigen ineffectively due to defective expression of B7 costimulatory cell surface molecules. J Clin Invest 1995; 95:1415-21.
53. Bailis W, Yashiro-Ohtani Y, Fang T C, Hatton R D, Weaver C T, Artis D, et al. Notch simultaneously orchestrates multiple helper T cell programs independently of cytokine signals. Immunity 2013; 39:148-59.
54. Hirota T, Takahashi A, Kubo M, Tsunoda T, Tomita K, Doi S, et al. Genome-wide association study identifies three new susceptibility loci for adult asthma in the Japanese population. Nat Genet 2011; 43:893-6.
55. Tindemans I, Peeters M J W, Hendriks R W. Notch Signaling in T Helper Cell Subsets: Instructor or Unbiased Amplifier? Front Immunol 2017; 8:419.
56. Komatsu N, Okamoto K, Sawa S, Nakashima T, Oh-hora M, Kodama T, et al. Pathogenic conversion of Foxp3+ T cells into TH17 cells in autoimmune arthritis. Nat Med 2014; 20:62-8.
57. Uyttendaele H, Closson V, Wu G, Roux F, Weinmaster G, Kitajewski J. Notch4 and Jagged-1 induce microvessel differentiation of rat brain endothelial cells. Microvasc Res 2000; 60:91-103.
58. Miniati D, Jelin E B, Ng J, Wu J, Carlson T R, Wu X, et al. Constitutively active endothelial Notch4 causes lung arteriovenous shunts in mice. Am J Physiol Lung Cell Mol Physiol 2010; 298:L169-77.

### Example 2

Notch Signaling in T helper cell differentiation. In immune cells, Notch is activated at many stages of development and differentiation of various T cell lineages^{2,3}. For instance, the activation of naive CD8⁺ T cells requires binding of DLL1 on antigen-presenting cells by Notch1 or Notch2, and leads to the expression of Eomes, Granzyme B and IFNγ Norch signaling also directs the differentiation of T helper (Th) cell subsets^{2,3}. In naive CD4⁺ T cells, DLL1 and DLL4 activate Notch signaling and transcription of Tbx21, which encodes the Th1 cell transcriptional regulator T-bet. During the differentiation of Th2 cells, activation of Notch1 and Notch2 by Jagged1 and Jagged2 favors the expression of GATA3 and IL-4. Notch1 signaling is reported to be important in the differentiation of the Th17 and Th9 subsets of helper T cells by promoting the expression of RORγt and IL-9^{4,5}.

Studies in mice have shown that inhibition of Notch processing, with γ-secretase inhibitors, or Notch function in CD4⁺ T cells with a dominant negative MAML1 reduces protective Th2 immunity against the gastrointestinal helminth, Trichuris muris and pulmonary allergic responses to allergen challenge as well as suppress Th1-mediated inflammation⁶. Additionally, recent studies have focused on the role of Notch signaling in Treg differentiation and function. Suppression of Notch signaling in Treg cells appears to drive a super regulatory phenotype and mice with targeted loss of RBPJ or Notch1 or Pofut1 in Foxp3⁺ T cells are protected from lethal GvHD. In contrast, Treg cells overexpressing a constructively active form of Notch1, N1c, appear to polarize the Treg cells to a more Th1-like phenotype, driving autoimmune phenotypes. The role of Notch signaling in negatively regulating allergic airway inflammation is largely unknown. As detailed below, our studies have uncovered a critical role for inducible Treg cell-specific expression of Notch4 in promoting airway inflammation. The characteristics of this pathway and its potential as a target of therapeutic intervention is the subject of this proposal.

Innovation

The discovery of a role for Notch4 signaling in airway inflammation defines a hitherto unappreciated novel pathway in disease pathogenesis that is promising as a target of therapeutic intervention in asthma. Furthermore, the demonstration, via data presented herein, that this pathway primarily acts to disable Treg cell function in airway inflammation indicates that its targeting will allow effective allergen-specific Treg cell responses and possible long term tolerogenic effects. Details on the scope of Notch4 role in human asthma and on how this pathway targets Treg cell subpopulations to disable their function are described herein.

Notch Signaling in Treg Cells Adversely Influences Airway Inflammation.

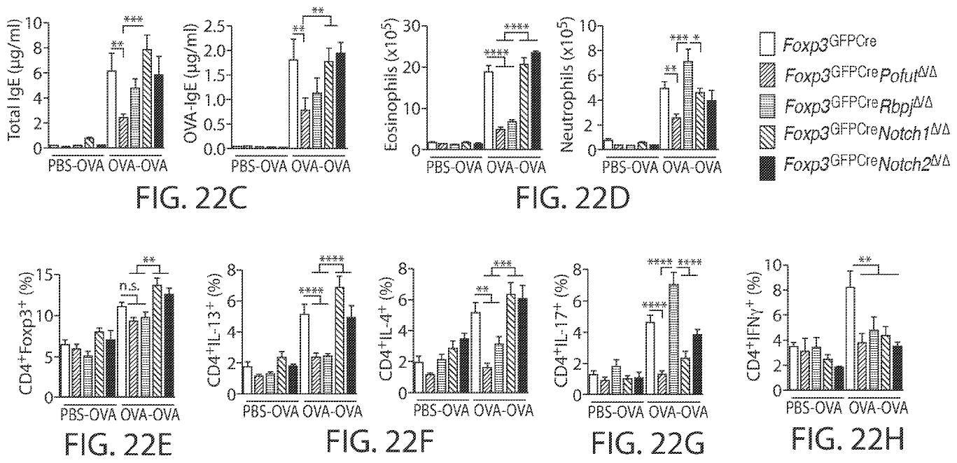

A negative function for Treg cell-intrinsic Notch signaling in in controlling peripheral tolerance was have previously described⁸. To determine the role of Notch signaling in Treg cells in airway inflammation, mice in which Notch signaling was specifically inactivated in Treg cells by cell lineage-specific deletion of the gene encoding the enzyme Pofut1 using a Foxp3 gene driven Cre recombinase (Foxp3^{GFPCre}Pofut1^{Δ/Δ}) were employed⁸. Pofut1 mediates o-fucosylation of Notch receptors, a requisite event in their glycosylation and essential to their function⁹. Its deficiency abrogates signaling via all Notch receptors¹⁰. Foxp3^{GFPCre}Pofut1^{Δ/Δ} mice sensitized and challenged with OVA exhibited markedly decreased tissue inflammation and airway hyper-responsiveness (AHR), lung tissue eosinophilia and neutorphilia, total and OVA specific IgE responses, and lung tissue Th2 and Th17 cell infiltration as compared to similarly sensitized and challenged Pofut1-sufficient control Foxp3^{GFPCre} mice (FIG. 22A-22H). Similar results were found using another Foxp3-driven Cre recombinase (Foxp3^{YFPCre}; data not shown).

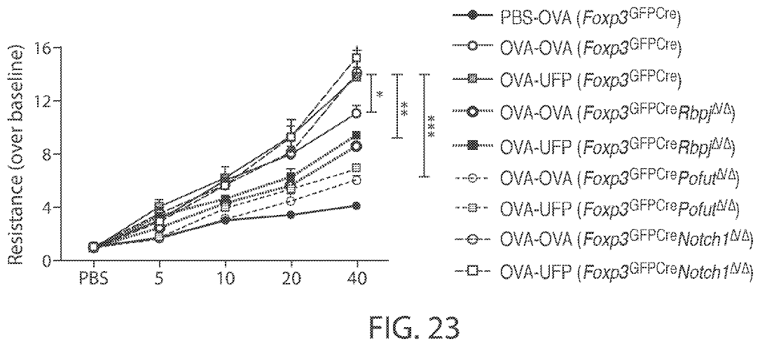

To determine the role of the canonical versus non-canonical pathways in mediating the effects of Notch signaling on Treg cells in airway inflammation, the impact of deleting Rbpj, encoding the canonical Notch factor RBPJ, in Treg cells on airway responses were examined^{8,11}. Results revealed that mice with Treg cell specific deletion of Rpbj (Foxp3^{YFPCre}Rpbj^{Δ/Δ}) exhibited an intermediate phenotype of decreased AHR and tissue eosinophilia in-between those of Foxp3^{Cre}Pofut1^{Δ/Δ} and Foxp3^{YFPCre} mice (FIG. 22A-22H). Significantly, RBPJ-deficient Treg cells suppressed airway eosinophilia but not neutrophilia and the Th2 but not the Th17 cell response, indicating a requirement for Treg cell-specific canonical (RBPJ-dependent) Notch signaling to control airway Th17 cell responses. The role of individual Notch receptors in modulating Treg cell function in airway inflammation was examined. Whereas it has previously been found that deletion of Notch1 in Treg cells enabled their regulation of Th1 responses⁸, Treg cell-specific deletion of floxed Notch1 or Notch1 alleles had no impact on allergic airway inflammation (FIG. 22A-22H). Thus, both Notch canonical and non-canonical pathways in Treg cells promote allergic airway inflammation, most likely via Notch3 and/or Notch4 signaling (see also FIG. 23).

Finally, Treg cell-specific Pofut1 deletion profoundly suppressed the exacerbation of OVA-induced allergic airway inflammation by UFP in a manner similar to that of OVA alone (FIG. 23), Again, Treg cell-specific Rbpj deletion gave an intermediate phenotype, while Notch1 deletion had no effect. Th cell cytokine expression in the respective mouse strains was similar to that observed in FIG. 22A-22H. These results indicated that activation of Notch signaling in Treg cells is a common mechanism for promoting airway inflammation shared by allergens and PM (FIG. 23)

Notch4 Controls the Treg Cell Response in Airway Inflammation.

Notch1/Notch2 appear to be the dominant receptors expressed on T cells, however the phenotype of Notch1 or Notch2 KO animals as well as the observed toxicity of g-secretase inhibitors has limited clinical intervention of these axes for inflammatory diseases. In contrast the phenotype of Notch4 KO mice is remarkably benign a few data are supported a compelling role for this receptor in development and physiology. Recent emerging data from the Chatila lab have indicated that in response to allergen challenge in the airway, alone or in synergy with ambient ultrafine particulate matter (UFP) generated by vehicular combustion engines OVA/UFP challenge Notch 4 expression appears to preferentially elevate on lung resident Treg cells, driving GATA3 expression and a Th2 cell-like phenotype of Treg cells that plays an essential role in airway inflammation.

It was previously shown that ultra fine particles (UFP) (and coarser fine particles as well) generated by combustion engines promote allergic airway inflammation by inducing Jagged 1 (Jag1) expression on antigen presenting cells in the lung¹². Jag1 in turn interacts with Notch receptors on allergen specific T cells to exacerbate allergic airway inflammation.

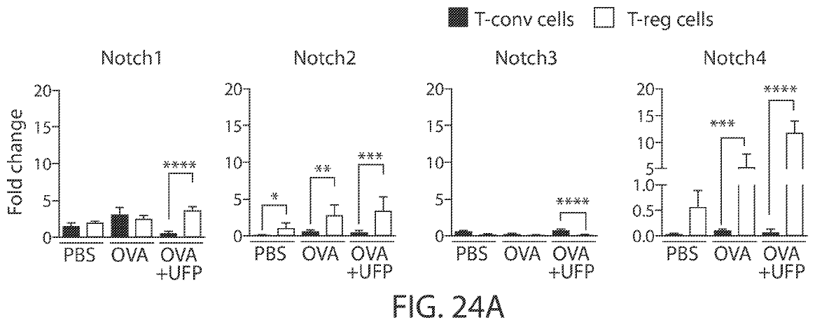

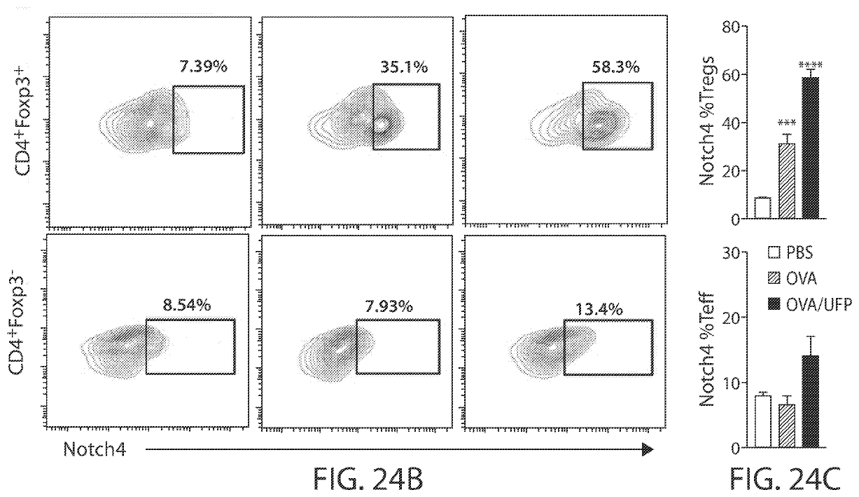

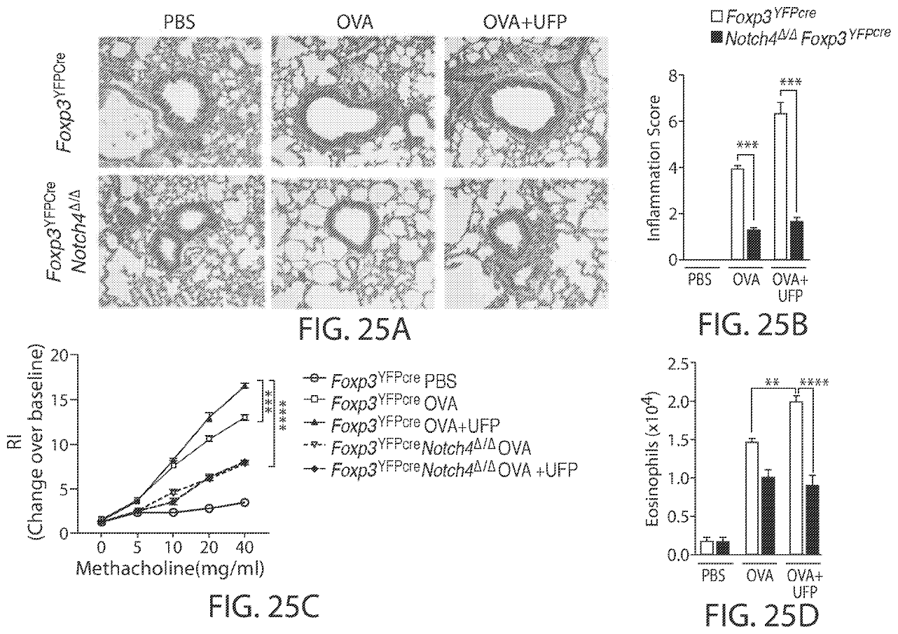

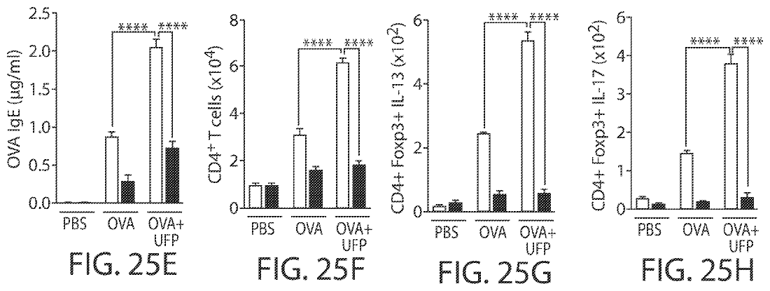

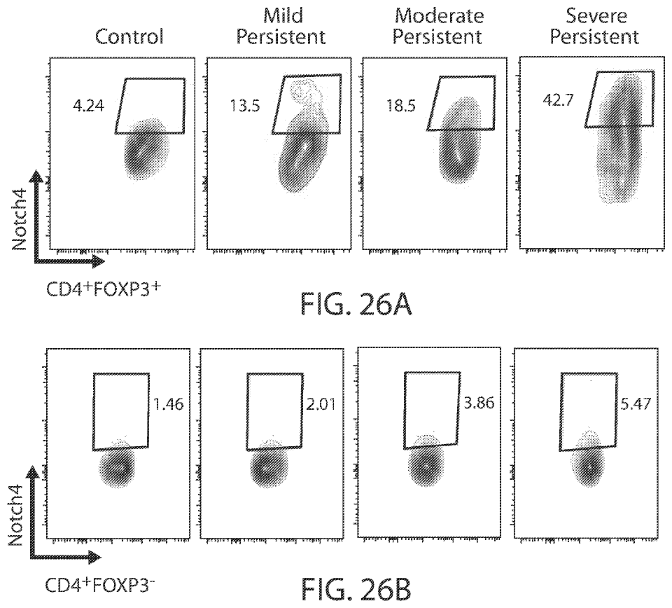

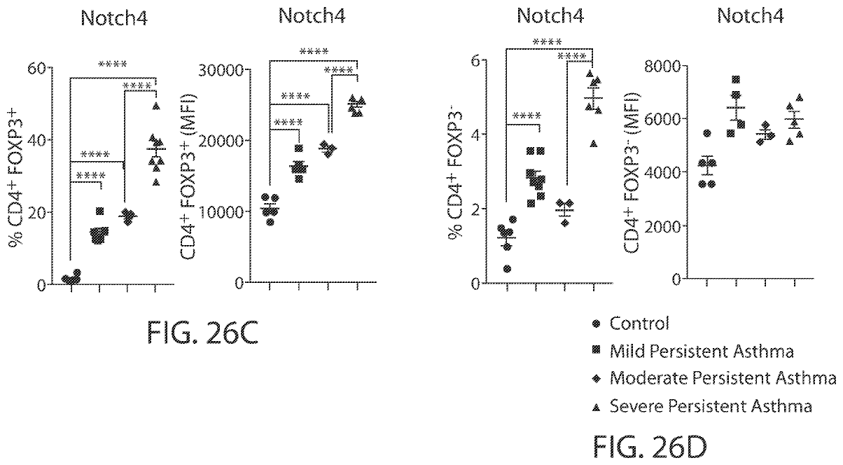

The identification of alveolar macrophages as the site of action of UFP was of particular interest as these cell types mediate the induction of allergen-specific Treg cells in the airways¹³. This finding suggested that corruption of Treg cells as the underlying mechanism for the exacerbation of the inflammatory response by UFP. It was shown that whereas deletion of Notch1 or Notch2 on Treg cells did not impact airway inflammation induced by sensitization and challenge with the allergen ovalbumin (OVA) either alone or together with UFP, deletion of Pofut1, a Notch fucosylating enzyme that is essential to their signaling function, abrogated almost completely both allergen and UFP-induced airway inflammation. The OVA model of airway inflammation was employed to examine Notch1-4 transcripts in cell-sorted lung resident CD4⁺Foxp3⁺ Treg cells and CD4⁺Foxp3⁻ Tconv cells of mice that were OVA-sensitized and challenged with OVA or OVA+UFP as compared to those that were sham sensitized. Results revealed that Notch4 transcripts were strikingly upregulated in lung resident CD4⁺Foxp3⁺ Treg cells but not CD4⁺Foxp3⁻ Tconv cells in mice that were OVA-sensitized and challenged either with OVA or especially OVA+UFP (FIG. 24A). In contrast, transcripts of other Notch receptors were either unchanged or marginally increased in comparison to those of Notch4 (data not shown). Staining with an anti-Notch4 mAb revealed that Notch4 expression on Treg cells was similarly upregulated in resident Treg cells but only marginally in resident Tconv cells of OVA and OVA+UFP treated mice (FIG. 24B-24C).

The functional significance of Notch4 upregulation in Treg cells in airway inflammation was examined using a genetic mouse model in which a foxed Notch4 allele was specifically deleted in Treg cells using a Foxp3-driven Cre recombinase (Foxp3^{YFPCre}) OVA-sensitized mice with deleted Notch4 in their Treg cells (Foxp3^{YFPCre}Notch4^{Δ/Δ}) exhibited a markedly attenuated airway inflammatory response when sensitized with OVA and then challenged with either OVA or OVA/UFP, with decreased airway resistance, tissue inflammation, eosinophilia and OVA-specific IgE responses as compared to mice with Notch4-sufficient Treg cells (FIG. 23). The attenuated airway inflammatory response in Foxp3^{YFPCre}Notch4^{Δ/Δ} mice was similar to what we previously have shown in the grant proposal for Foxp3^{YFPCre}Pofut4^{Δ/Δ} mice, whose Notch signaling in Treg cells is abrogated due to the deficiency of the fucosylating enzyme Pofut1, indicating that signaling via Notch4 accounted for most If not all of the immune dysregulatory effects of Notch signaling in Treg cells in airway inflammation. Significantly, airway inflammation induced by OVA or OVA/UFP was brought down to the same level upon Notch4 deletion in Treg cells, suggesting that Notch4 signaling is a common pathway relevant to both allergen- and UFP-mediated airway inflammation. While Jag1 deletion in alveolar macrophages abrogated the augmentation of airway inflammation by UFP, it had very modest effects on allergen induced airway inflammation, suggesting that different Notch ligands, one induced by OVA whose identify is currently being investigated, and the other being Jag1 induced by UFP, interact with Notch4 on Tre cells to mediate airway inflammation.

Overall, these results presented herein indicate that Notch4 is a critical pathway for driving allergic inflammation that is common to both allergens and ambient particulate matter pollutants, and that it acts by effecting plastic changes to tissue Treg cells, leading to loss of tolerance to allergens. Furthermore, preliminary data on pediatric severe asthma patients indicated that Notch4 expression was elevated on their peripheral blood Treg cells as compared to those of normal healthy controls, indicating a spill-over effect that can be monitored in asthmatics both as a biomarker of disease and for therapeutic purposes. Taken together, these data suggest that in targeting Notch4 might be an approach to restoring normal airways homeostasis in patients with asthma and possibly extending to other diseases such as chronic obstructive pulmonary disease (COPD).

### REFERENCES

1. Siebel, C. & Lendahl, U. Notch Signaling in Development, Tissue Homeostasis, and Disease. Physiol Rev 97, 1235-1294 (2017).
2. Amsen, D., Helbig, C. & Backer, R. A. Notch in T Cell Differentiation: All Things Considered. Trends Immunol 36, 802-814 (2015).
3. Tindemans, I., Peeters, M. J. W. & Hendriks, R. W. Notch Signaling in T Helper Cell Subsets: Instructor or Unbiased Amplifier? Frontiers in immunology 8, 419 (2017).
4. Meyer Zu Horste, G., et al. RBPJ Controls Development of Pathogenic Th17 Cells by Regulating IL-23 Receptor Expression. Cell reports 16, 392-404 (2016).
5. Elyaman, W., et al. Notch receptors and Smad3 signaling cooperate in the induction of interleukin-9-producing T cells. Immunity 36, 623-634 (2012).
6. Tu, L., et al. Notch signaling is an important regulator of type 2 immunity. J Exp Med 202, 1037-1042 (2005).
7. Hirota, T., et al. Genome-wide association study identifies three new susceptibility loci for adult asthma in the Japanese population. Nat Genet 43, 893-896 (2011).
8. Charbonnier, L. M., Wang, S., Georgiev, P., Sefik, E. & Chatila, T. A. Control of peripheral tolerance by regulatory T cell-intrinsic Notch signaling. Nat Immunol 16, 1162-1173 (2015).
9. Stanley, P. & Guidos, C. J. Regulation of Notch signaling during T- and B-cell development by 0-fucose glycans. Immunol Rev 230, 201-215 (2009).
10. Shi, S. & Stanley, P. Protein 0-fucosyltransferase 1 is an essential component of Notch signaling pathways. Proc Natl Acad Sci USA 100, 5234-5239 (2003).
11. Han, H., et al. Inducible gene knockout of transcription factor recombination signal binding protein-J reveals its essential role in T versus B lineage decision. Int Immunol 14, 637-645 (2002).
12. Xia, M., et al. Vehicular exhaust particles promote allergic airway inflammation through an aryl hydrocarbon receptor-notch signaling cascade. J Allergy Clin Immunol (2015).
13. Soroosh, P., et al. Lung-resident tissue macrophages generate Foxp3+ regulatory T cells and promote airway tolerance. J Exp Med 210, 775-788 (2013).

What is claimed is:

1) A method for treating or preventing asthma or an allergic disease, comprising administering to a subject having asthma or an allergic disease an effective amount of an agent that inhibits Notch4.

2) The method of claim 1, further comprising, prior to administering,
identifying a subject as having asthma or an allergic disease.

3) The method of claim 1, wherein the asthma is selected from the list consisting of allergic asthma, asthma without allergies, aspirin exacerbated respiratory disease, exercise induced asthma, cough variant, and occupational asthma.

4) The method of claim 1, wherein the allergic disease is selected from the list consisting of allergic rhinitis, sinusitis, otitis media, atopic dermatitis, urticaria, angioedema, and anaphylaxis.

5) The method of claim 1, wherein the agent that inhibits Notch4 is selected from the group consisting of a small molecule, an antibody, a peptide, a genome editing system, an antisense oligonucleotide, and an RNAi.

6) The method of claim 5, wherein the antibody is a humanized antibody.

7) The method of claim 5, wherein the RNAi is a microRNA, an siRNA, or a shRNA.

8) The method of claim 1, wherein inhibiting Notch4 is inhibiting the expression level and/or activity of Notch4.

9) The method of claim 8, wherein the expression level and/or activity of Notch4 is inhibited by at least 50%, at least 60%, at least 70%, at least 80%, at least 90%, or more as compared to an appropriate control.

10) The method of claim 1, wherein Notch4 is inhibited on T regulatory cells.

11) The method of claim 1, further comprising administering at least one additional anti-asthma therapeutic.

12) The method of claim 1 and 2, further comprising administering at least one additional anti-allergic disease therapeutic.

13) (canceled)

14) The method of claim 1, further comprising, prior to administering, identifying a subject at risk of having asthma or an allergic disease.

15) A composition for the treatment of asthma or an allergic disease, the composition comprising an agent that inhibits Notch4 and a pharmaceutically acceptable carrier.

16)-18) (canceled)

19) The composition of claim 15, wherein the composition is formulated for inhaled administration.

20) A method for treating a subject at risk of having asthma or an allergic disease, the method comprising,
a. Obtaining a biological sample from the subject;
b. measuring the level of Notch4 in a population of candidate cells;
wherein the subject is at risk of having asthma or an allergic disease if the level of Notch is increased as compared to a reference level; and
c. administering an agent that inhibits Notch4 to a subject at risk.

21) The method of claim 20, wherein the level of Notch4 is increased by at least 2-fold, at least 3-fold, at least 4-fold, at least 5-fold, at least 6-fold, at least 7-fold, at least 8-fold, at least 9-fold, at least 10-fold, or more as compared to a reference level.

22) (canceled)

23) (canceled)
